SEQUENCE LISTING (6 sequences)

SEQ ID NO: 1 - protein, 14 aa, synthetic construct
CRXRRXRRXRRXRC

SEQ ID NO: 2 - protein, 13 aa, synthetic construct
RXRRXRRXRRXRC

SEQ ID NO: 3 - protein, 14 aa, synthetic construct
RXRRXRCRXRCRXR

SEQ ID NO: 4 - protein, 12 aa, synthetic construct
RXRRXRRXRRXR

SEQ ID NO: 5 - protein, 6 aa, synthetic construct
RXRRXR

SEQ ID NO: 6 - protein, 9 aa, synthetic construct
RXRRXRRXR

(19) United States
(12) Patent Application Publication
(10) Pub. No.: US 2024/0368226 A1
(43) Pub. Date: Nov. 7, 2024

(54) Cyclic Cell Penetrating Peptides For Treating Neisseria Gonorrhoeae Infections

(71) Applicants: The United States Government As Represented By The Department Of Veterans Affairs, Washington, DC (US); The Regents of the University of California, Oakland, CA (US)

(72) Inventors: Constance John, San Francisco, CA (US); Gary Jarvis, San Francisco, CA (US)

(21) Appl. No.: 18/655,023

(22) Filed: May 3, 2024

Related U.S. Application Data
(60) Provisional application No. 63/499,853, filed on May 3, 2023.

(51) Int. Cl. C07K 7/54; A61K 38/00; A61P 31/04

(52) U.S. Cl. CPC C07K 7/54; A61P 31/04; A61K 38/00

(57) ABSTRACT

Disclosed herein are cyclic peptides having activity as cell penetrating peptides (CPPs). Disclosed are cyclic cell penetrating peptides (cCPPs) comprising one or more of the cell penetrating peptides disclosed herein. Disclosed are cCPPs comprising a 6-aminohexanoic-spaced oligoarginine. Disclosed are cCPPs comprising the amino acid sequence of CRXRRXRRXRRXRC (SEQ ID NO:1), wherein X is aminohexanoic acid. In some aspects, the cCPP comprises a disulfide bridge. Disclosed are cCPPs comprising the amino acid sequence of RXRRXRRXRRXRC (SEQ ID NO:2), wherein X is aminohexanoic acid. In some aspects, the cCPPs comprise a thioether bridge.

### CROSS-REFERENCE TO RELATED APPLICATIONS

This application claims the benefit of U.S. Provisional Patent Application No. 63/499,853, filed May 3, 2023, which is incorporated by reference herein in its entirety.

### REFERENCE TO SEQUENCE LISTING

The Sequence Listing submitted May 3, 2024 as a text file named “37759.0545U2.xml,” created on May 3, 2024, and having a size of 6,125 bytes is hereby incorporated by reference pursuant to 37 C.F.R. § 1.52 (c) (5).

### BACKGROUND

Infections with N. gonorrhoeae are a major cause of morbidity with an estimated 1.6 million cases in the U.S. and 87 million cases worldwide annually. There is a great need for new antibiotics due to the alarming rise in multidrug-resistant (MDR) strains, which is making emergence of untreatable gonococcal infections a real prospect. Clinical isolates resistant to the first-line antibiotic, ceftriaxone, have been reported in countries including Denmark, Canada and Japan underlying a compelling need for new antimicrobials. Importantly, a growing number of studies have shown that gonococcal infection can facilitate the transmission of HIV. Further, control of gonococcal infection is widely recognized as increasingly challenging due to lack of any vaccine. As such, MDR N. gonorrhoeae is classified by CDC as an urgent public health threat that requires aggressive action.

### BRIEF SUMMARY

Disclosed are approaches to treating gonococcal infections given the disturbing increase in MDR organisms. Disclosed herein are cell penetrating peptides (CPPs) for gonococci. The disclosed CPPs can be bactericidal for gonococci, can reduce the induction of cytokines in human cells in response to gonococcal infection, and can be non-cytotoxic for human cells. In order to increase the potency and selectivity of CPPs, disclosed are structural variants of CPPs. The disclosed CPPs have cyclic structures unlike prior linear CPPs. Using bactericidal assays as a measure of potency, the disclosed CPPs are significantly more potent than prior identified linear CPPs.

Disclosed herein are cyclic peptides having activity as cell penetrating peptides.

Disclosed are cyclic cell penetrating peptides (cCPPs) comprising one or more of the cell penetrating peptides disclosed herein.

Disclosed are cCPPs comprising a 6-aminohexanoic-spaced oligoarginine.

Disclosed are cCPPs comprising the amino acid sequence of CRXRRXRRXRRXRC (SEQ ID NO:1), wherein X is aminohexanoic acid. In some aspects, the cCPP comprises a disulfide bridge.

Disclosed are cCPPs comprising the amino acid sequence of RXRRXRRXRRXRC (SEQ ID NO:2), wherein X is aminohexanoic acid. In some aspects, the cCPPs comprise a thioether bridge.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1C show structures of the three CPP variants. (A): Structure of linear 12 amino acid CPP RXRRXRRXRRXR (MW 1720.1). (B): Structure of cyclic 14 amino acid CPP CRXRRXRRXRRXRC—NH2 (SEQ ID NO:1) (disulfide bridge C1-C2; MW 1923.4). (C): Structure of cyclic 13 amino acid CPP c [RXRRXRRXRRXRC(S—)] (SEQ ID NO:2) that is cyclized by a bond between the sulfur in Cys at position 13 and a N-terminal acetate group (thioether cyclic; MW 1862.3). R=arginine and X=6-aminohexanoic acid.

FIGS. 2A-2C shows bactericidal activity of linear and cyclic CPP for human challenge gonococcal strain FA1090 and gonococcal MDR strains F89 and H041. Increasing concentrations of CPP were incubated with gonococci for 4 h, and survival was determined. Survival of all three strains was significantly reduced in the presence of the CPP with greater killing by the cyclic CPP. Bars represent the mean±SEM of triplicate experiments that were each performed in duplicate. (A): Linear 12 amino acid CPP. (B): Cyclic disulfide CPP. (C): Cyclic thioether CPP.

FIGS. 3A and 3B show timelines for Pk blood collection (A) and in vivo efficacy testing in the female mouse model of gonorrhea (B).

FIG. 4 is a table showing the treatment and experimental groups for the in vivo efficacy testing in a female mouse model.

FIG. 5 is a table of cell penetrating peptides that target bacteria.

FIG. 6 is a table of cell penetrating peptides that target viruses.

FIG. 7 is a table of cell penetrating peptides that target parasites.

FIGS. 8A-8C show bactericidal activity of increasing concentrations (0.8, 4, 20, and 100 μM) of the linear CPP, DS (disulfide) cyclized CPP (DS), and TE (thioether) cyclized CPP (TE) for the FA1090 1-81-S2 (A), F89 (B) and H041 gonococcal isolates (C) after incubation with the bacteria in RPMI 1640 cell culture medium with 10% FBS for 4 h. Bars in order: CPP, DS, and TE that each represent the mean±SEM from three experiments that were each performed in triplicate. **P<0.01, ***P<0.001.

FIGS. 9A-9E show relative levels of LDH in culture supernatants of human End1/E6E7 (A) ME-180 (B) and activated THP-1 cells (C) in the presence of the linear, DS, and TE cyclized CPPs (0, 100, and 200 μM), and in culture supernatants of End1/E6E7 and ME-180 cells (D) in the presence of LL-37 (0, 20, and 40 μM). Percentage of hemolysis of sheep RBCs (E) from DMSO vehicle control (DO at 0.5%, 2.0% DO), linear CPP (LR at 100 mM, 200 mM), DS cyclized CPP (at 100 mM, 200 mM), TE cyclized CPP (at 100 mM, 200 mM) and Triton-X-100 control (TX, 0.1%). Bars represent the mean±SEM of three experiments that were each performed in triplicate. *P<0.05, **P<0.01, ***P<0.001.

FIGS. 10A-10C show bactericidal activity of increasing concentrations (0.4, 20, and 100 mM) of linear and TE cyclized CPPs for the FA1090 1-81-S2 (A), F89 (B) and H041 (C) gonococcal isolates grown on GC agar containing CMP-NANA (10 μg/mL). Bars represent the mean±SEM from three experiments that were each performed in triplicate. Bar represents the mean±SEM. ***P<0.001.

FIGS. 11A-11C show bactericidal activity of increasing concentrations (4, 20 and 100 mM) of linear and TE cyclized CPPs for FA10901-81-S2 gonococci in the presence of End1/E6E7 endocervical epithelial cells (A), ME-180 cervical epithelia cells (B), and THP-1 monocytes (C) in 180 μL of RPMI-1640 media with 10% FBS. Bars represent the mean±SEM from three experiments that were each performed in triplicate. *P<0.05, **P<0.01, ***P<0.001.

FIGS. 12A-12C show levels of TNF-α in supernatants of activated human THP-1 cells infected with gonococcal isolates FA1090 (A), F89 (B), and H041 (C) at a moi of 1 in the presence of increasing concentrations of the linear and TE cyclized CPP (0, 4, 20, and 100 μM) overnight. Bars represent the mean±SEM from three experiments each performed with three or more replicates. ***P<0.001.

FIGS. 13A-13C show bactericidal activity of increasing concentrations (0.8, 4, 20, and 100 mM) of linear and TE cyclized CPPs for N. flavescens strain 4322 (A), N. lactamica 1934 (B), and N. subflava strain 52 (C) after incubation with the bacteria in RPMI-1640 media with 10% FBS for 4 h. Bars represent the mean±SEM of triplicate experiments that were each performed in triplicate. *P<0.05, ***P<0.001.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cCPP” includes a plurality of such cCPPs, reference to “the cCPP” is a reference to one or more cCPP and equivalents thereof known to those skilled in the art, and so forth.

By “treat” is meant to administer a peptide or composition of the invention to a subject, such as a human or other mammal (for example, an animal model), that has an increased susceptibility for developing a disease, disorder or infection in order to prevent or delay onset of the disease disorder or infection, prevent or delay a worsening of the effects of the disease, disorder or infection, or to partially or fully reverse the effects of the disease, disorder or infection. In some aspects, treat can mean to ameliorate a symptom of a disease, disorder or infection. In some aspects, the disease, disorder or infection is N. gonorrhoeae.

By “prevent” is meant to minimize the chance that a subject who has an increased susceptibility for developing a disease, disorder or infection will actually develop the disease, disorder or infection.

As used herein, the term “subject” or “patient” can be used interchangeably and refer to any person to which a peptide or composition of the invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are humans as N. gonorrhoeae typically infects only humans.

As used herein, the terms “administering” and “administration” refer to any method of providing a disclosed peptide or composition of the invention to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed exosome so as to treat a subject.

The terms “variant” and “mutant” can be used interchangeably herein. As used herein, the term “variant” refers to a modified amino acid or amino acid sequence which displays the same characteristics when compared to a reference nucleic acid or protein sequence. A variant can be at least 65, 70, 75, 80, 85, 90, 95, or 99 percent homologous to a reference sequence. In some aspects, a reference sequence can be a fragment of one or more of the disclosed sequences. A “variant” can mean a difference in some way from the reference sequence other than just a simple deletion of an N- and/or C-terminal amino acid. A variant can also be a difference in the nucleotide sequence. Variants can also, or alternatively, include at least one substitution and/or at least one addition; there may also be at least one deletion. Alternatively or in addition, variants can comprise modifications, such as non-natural residues at one or more positions with respect to a reference nucleic acid or protein.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Cyclic CPPs

Disclosed herein are cyclic peptides having activity as cell penetrating peptides (CPPs). Disclosed are cyclic cell penetrating peptides (cCPPs) comprising one or more of the cell penetrating peptides disclosed herein, or a derivative thereof. In some aspects, a cCPP is a linear cell penetrating peptide sequence that has been cyclized head-to-tail to form a cyclic structure.

Disclosed are cCPPs comprising a 6-aminohexanoic-spaced oligoarginine. In some aspects, the 6-aminohexanoic-spaced oligoarginine is (R-Ahx-R)₄. In some aspects, the 6-aminohexanoic-spaced oligoarginine can be, but is not limited to, (R-Ahx-R)₂. (R-Ahx-R)₃. (R-Ahx-R)₄. (R-Ahx-R)₅. (R-Ahx-R)₆. In some aspects, cCPPs can be a variant of the 6-aminohexanoic-spaced oligoarginines disclosed herein. In some aspects, a variant can be 70, 75, 80, 85, 90, 95, or 99% identical to the cCPPs disclosed herein. For example, in some aspects, a variant cCPP can comprise a Lys substitution for one or more of the arginines in the disclosed cCPPs. In some aspects, a variant cCPP can comprise a conservative or non-conservative amino acid substitution for one or more of the arginines in the disclosed cCPPs.

In some aspects, the cCPPs further comprise cysteines on the N- and C-terminal ends. In some aspects, the cCPPs comprise a disulfide bridge.

In some aspects, the cCPPs comprise a thioether bridge.

Disclosed are cCPPs comprising the amino acid sequence of CRXRRXRRXRRXRC (SEQ ID NO:1), wherein X is aminohexanoic acid. In some aspects, the cCPP comprises a disulfide bridge.

Disclosed are cCPPs comprising the amino acid sequence of RXRRXRRXRRXRC (SEQ ID NO:2), wherein X is aminohexanoic acid. In some aspects, the cCPPs comprise a thioether bridge. For example, a cCPP comprising a thioether bridge can correspond to c [RXRRXRRXRRXRC(S—)] (SEQ ID NO:2) and it can be cyclized by a sulfide bond between the sulfur in Cys at position 13 and a N-terminal acetate group.

In some aspects, the cCPPs can comprise a total number of amino acids in the range of from 4 to about 20 amino acids. In some embodiments, the cCPPs disclosed herein comprise about 4 to about to about 13 amino acids, e.g., about 5, about 6, about 7, about 8, about 9, about 10, or about 11 amino acids, about 12 amino acids, about 13 amino acids, or about 14 amino acids, inclusive of all ranges and subranges there between. In some aspects, the cCPPs disclosed herein comprise from about 5 to about 15 amino acids, from about 6 to about 10 amino acids, or from about 6 to about 8 amino acids.

In some aspects, each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, alloisoleucine, arginine, asparagine, aspartic acid, cysteine, glutamine, glutamic acid, glycine, histidine, isoleucine, leucine, lysine, methionine, naphthylalanine, phenylalanine, proline, pyroglutamic acid, serine, threonine, tryptophan, tyrosine, valine, a derivative, or combinations thereof. Analogs of amino acids encompass those that have a structural similarity but are not identical to an amino acid, e.g., due to a modification to the side chain or backbone on said amino acid. Such modifications may increase the hydrophobicity of the side chain, including elongation of the side chain by one or more hydrocarbons, or increasing the solvent accessible surface area of an amino acid having an aromatic ring on its side chain, e.g., by conjugating a second aromatic ring or increasing the size of the aromatic ring. Derivatives of amino acids encompass natural and non-natural amino acids that have been modified (e.g., by substitution) to include a hydrophobic group as described herein. For example, a derivative of lysine includes lysine whose side chain has been substituted with alkylcarboxamidyl.

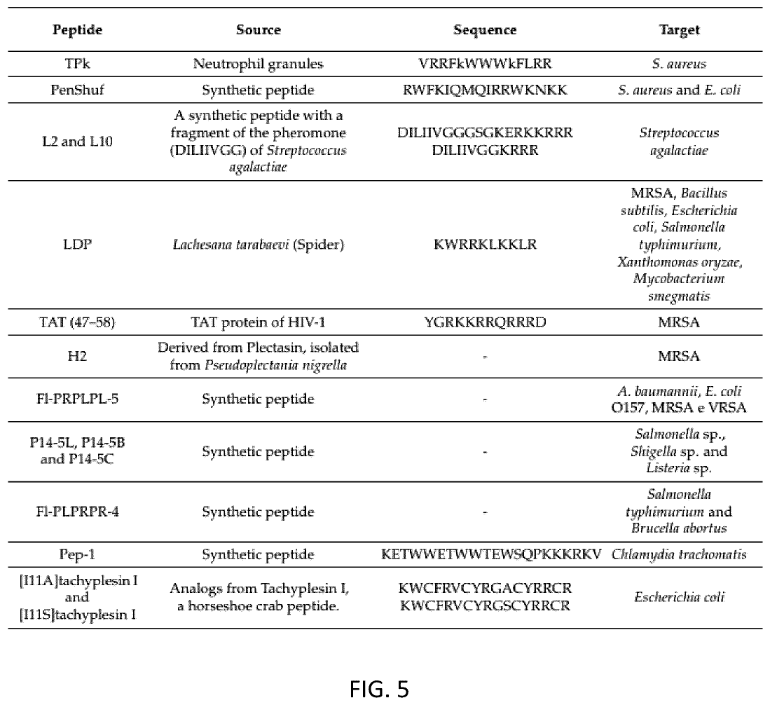

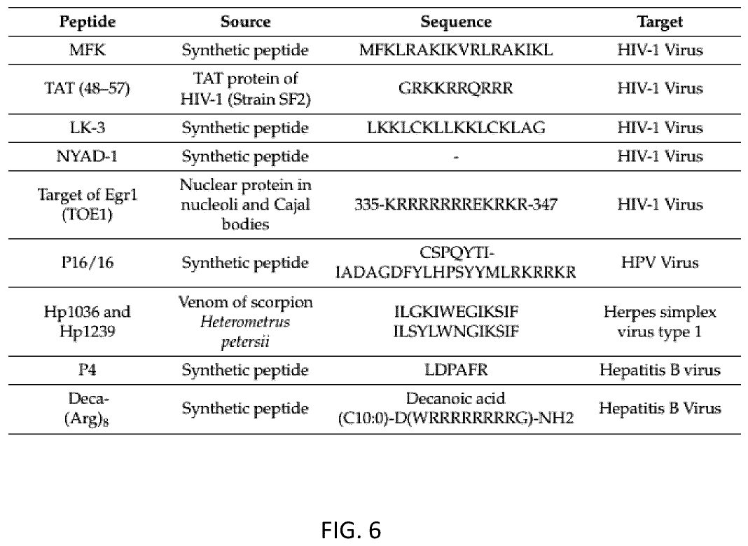

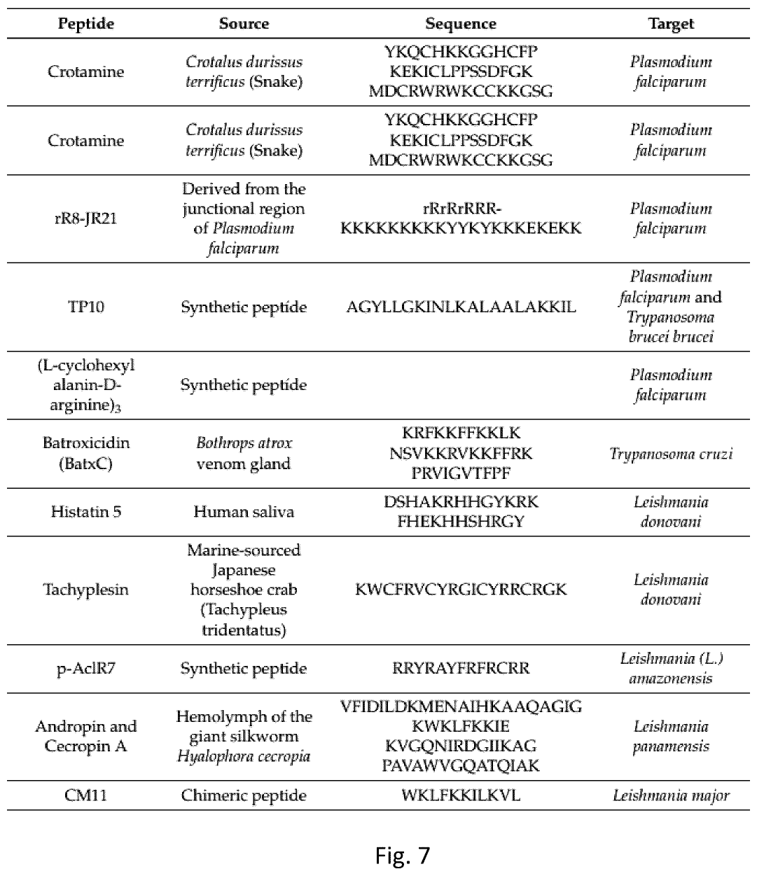

In some aspects, the cCPPs comprise a CPP having the sequence of one or more of those CPPs found in the CPPsite database (Agrawal et al. Nucleic Acids Research, 2016, vol 44, D1098-D1103; hereby incorporated by reference in its entirety as it pertains to the CPPsite database). Thus, in some aspects, the CPP is one or more of those described at http://crdd.osdd.net/raghava/cppsite/. In some aspects, the CPP can be any CPP listed in FIGS. 5-7.

### C. Compositions

Disclosed are compositions comprising any of the disclosed peptides. For example, disclosed are compositions comprising a cCPP comprising one or more of the cell penetrating peptides disclosed herein. Disclosed are compositions comprising a cCPP comprising the amino acid sequence of CRXRRXRRXRRXRC (SEQ ID NO:1) or RXRRXRRXRRXRC (SEQ ID NO:2), wherein X is aminohexanoic acid, or a variant thereof.

The disclosed pharmaceutical compositions can further comprise a pharmaceutically acceptable carrier.

### 1. Delivery of Compositions

In the methods described herein, delivery (or administration) of the disclosed peptides and compositions disclosed herein to cells or a subject can be via a variety of mechanisms. As defined above, disclosed herein are compositions comprising any one or more of the disclosed peptides and can also include a carrier such as a pharmaceutically acceptable carrier. For example, disclosed are pharmaceutical compositions, comprising a cCPP comprising the amino acid sequence of CRXRRXRRXRRXRC (SEQ ID NO:1) or RXRRXRRXRRXRC (SEQ ID NO:2), wherein X is aminohexanoic acid as disclosed herein and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG: PC: Cholesterol: peptide or PC: peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, glucose, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The disclosed delivery techniques can be used not only for the disclosed compositions but also the disclosed peptides.

In some aspects, the disclosed compositions and peptides are administered in combination with one or more additional agents. In some aspects, the additional agent can be, but is not limited to, a traditional therapeutic for the disease or disorder being treated. For example, a traditional therapeutic can be, but is not limited to, a therapeutic that treats an N. gonorrhoeae infection.

### D. Methods

Disclosed are methods of treating a subject having a N. gonorrhoeae infection comprising administering one or more of the disclosed cCPPs to the subject. In some aspects, the cCPPs can have a disulfide bond or thioether bond.

Disclosed are methods of killing N. gonorrhoeae comprising administering one or more of the disclosed cCPPs to the N. gonorrhoeae. In some aspects, the N. gonorrhoeae is in vitro or in vivo. Thus, in some aspects administering can mean to a subject infected with N. gonorrhoeae or to the bacteria itself (e.g., in culture).

In some aspects, the methods further comprise administering a therapeutic known to counter Gram-negative bacterial infections. In some aspects, the therapeutic is known to counter N. gonorrhoeae infections. In some aspects, the therapeutic can be, but is not limited to, gentamicin, azithromycin, cefixime, spectinomycin, or a combination thereof.

Disclosed are methods of reducing inflammatory signaling from cells infected with N. gonorrhoeae comprising administering one or more of the cCPPs of any one of claims 1-8 to the cells infected with N. gonorrhoeae. In some aspects, reducing inflammatory signaling comprises reducing the amount or levels of TNF-α produced by the cells infected with N. gonorrhoeae. In some aspects, the cells can be macrophage/monocytes. Thus, in some aspects, the disclosed cCPPs can have anti-inflammatory properties.

In some aspects of the disclosed methods, the disclosed cCPPs are bactericidal but not cytotoxic to the cells.

In some aspects, the cCPPs are administered subcutaneously, intravenously, topically, orally, opthalmically orintravaginally.

In some aspects, the cCPPs can have a disulfide bond or thioether bond. In some aspects, the cCPP can be, but is not limited to, CRXRRXRRXRRXRC (SEQ ID NO:1) or RXRRXRRXRRXRC (SEQ ID NO:2), wherein X is aminohexanoic acid.

### E. Dosages

Disclosed are dosing regimens comprising administering a single dose of one or more of the disclosed compositions or peptides to a subject in need thereof, wherein the single dose comprises an amount effective to kill N. gonorrhoeae.

Disclosed are dosing regimens comprising administering at least two doses of one or more of the disclosed compositions or peptides to a subject in need thereof, wherein each dose is the same concentration. In some aspects, each dose after a first dose can be decreased. In some aspects, each dose after a first dose can be increased.

In some aspects, a single dose can be a continuous administration. In some aspects, a continuous administration can be hours, days, weeks, or months. In some aspects, there can be two or more doses. In some aspects, the two or more doses can be administered days, weeks, or months apart.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of the disclosed peptides or compositions.

### EXAMPLES

### A. Example 1

### 1. General Purpose

Infections due to Neisseria gonorrhoeae are a major cause of morbidity with an estimated 1.6 million cases in the U.S. and 87 million cases worldwide annually. Within the VA Health Care System, the incidence of gonorrhea increased between 2013 and 2017 with the total number of cases in that time period at 10,587. N. gonorrhoeae has quickly developed resistance to all but one class of antibiotics and the rapid emergence of multidrug-resistant (MDR) strains could result in untreatable gonorrhea due to resistance that is developing to the third-generation cephalosporins, ceftriaxone and cefixime, which are recommended first line treatments. Importantly, a growing number of studies have shown that gonococcal infection can facilitate the transmission of HIV. Further, control of gonococcal infection is widely recognized as increasingly challenging due to lack of any vaccine. As such, MDR N. gonorrhoeae is classified by CDC as an urgent public health threat that requires aggressive action.

There is currently significant interest in cationic cell-penetrating peptides (CPP) as antibacterial agents particularly due to the alarming rise of MDR Gram-negative bacteria. The bactericidal potential of a linear 12 amino acid CPP was evaluated for MDR and human challenge strains of N. gonorrhoeae and the CPP penetrated the bacterial membrane and was bactericidal for all nine multi-drug resistant and human challenge strains of gonococci tested. Importantly, no apparent resistance to the CPP developed in surviving bacteria as susceptibility was the same in bacteria from colonies after exposure to CPP and then retreated. Further, the CPP reduced inflammatory cytokine induction and prevented bacterial cell invasion of cervical epithelial cells in the absence of measurable cell cytotoxicity.

### 2. Background

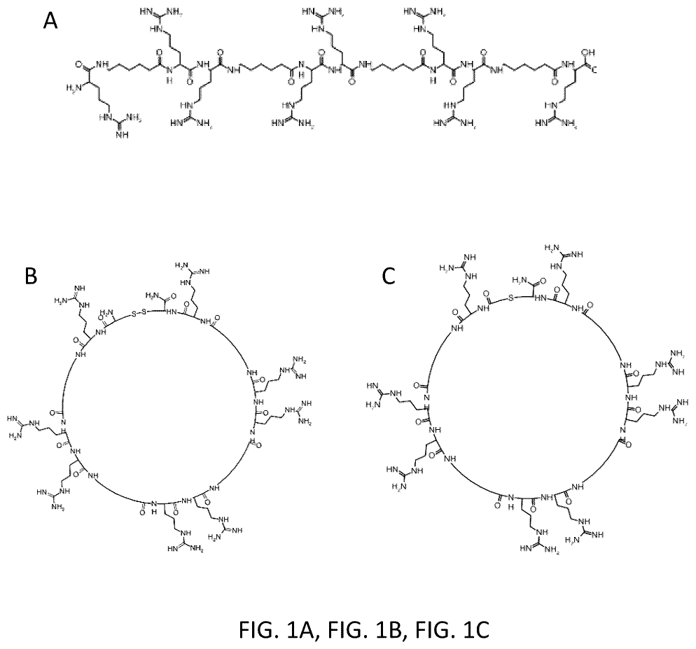

Most anti-microbial peptides (AMPs) such as human LL-37 have several generalized features in common, amphipathicity, mean hydrophobicity and overall cationic charge. The cationic charge of AMPs is thought to be critical for selectivity because bacterial membranes are comprised of mainly negatively charged molecules such as phosphatidylglycerol, cardiolipid, lipoteichoic acids, or lipopolysaccharide, whereas the outer leaflet of the membranes of mammalian cells contain more neutral molecules, such as sphingomyelin, phosphatidylcholine, and sterols. Compared to lysine, the other positively charged amino acid, arginine has been found to be preferable in CPPs as a molecular transporter into cells because of its ability to form two hydrogen bonds with anionic cell surface molecules. This feature should increase its selectivity for bacterial compared to mammalian cells. In addition to increasing hydrophobicity and flexibility, the presence of 6-aminohexanoic acid residues in a peptide otherwise consisting only of arginine residues as in (RXR)₄ (SEQ ID NO:4) is thought to increase serum and intracellular stability, possibly due to reduced susceptibility to proteolysis. A CPP, which has a linear structure (RXR)₄ (SEQ ID NO: 4) where X is aminohexanoic acid and R is arginine (FIG. 1A), was used for human challenge and antibiotic-resistant strains of N. gonorrhoeae in vitro. It was found that the linear CPP was bactericidal for gonococci, reduced inflammatory cytokine induction in cells, and prevented bacterial cell invasion in the absence of human cell cytotoxicity. The lack of human cell cytotoxicity contrasted with human LL-37 which exhibited cell cytotoxicity at the same doses of CPP.

### 3. Results

The strategy that was used to increase the potency and selectivity of the linear CPP was to cyclize it, which has been shown to be an approach that can constrain secondary structure, increase stability, and reduce the cytotoxicity of AMPs for mammalian cells. For example, a study focused on screening phage display peptide libraries showed that cyclization of two peptides decreased their minimum bactericidal concentration by 30-40-fold. Initially we synthesized and tested a cyclized form of the CPP created through formation of disulfide bonds that are cleavable in vivo, by use of an (RXR)₄ (SEQ ID NO:4) peptide containing two cysteines with one cysteine at each end, i.e., CRXRRXRRXRRXRC (SEQ ID NO:1) (FIG. 1B). The disulfide cyclic CPP was produced using Fmoc solid phase peptide synthesis. by the addition of a cysteine on each terminus followed by oxidation by incubation with iodine (I2) and, thus, had a structure corresponding to cyclo [C(S—)—R—XR—(R—X—R)2-R—X—R—C(S—)]—NH2 (SEQ ID NO:1) where S is sulfur. The thiocther CPP was formed via a thiocther bond and had a structure corresponding to cyclo [R—X—R—(R—X—R)2-R—C(S—)]—NH₂ (SEQ ID NO:2), where S is sulfur, by reaction of an N-terminal bromoacetyl group with a C-terminal cysteine. The structure of the CPP c [RXRRXRRXRRXRC(S—)] (SEQ ID NO:2) thioether cCPP is shown in FIG. 1C.

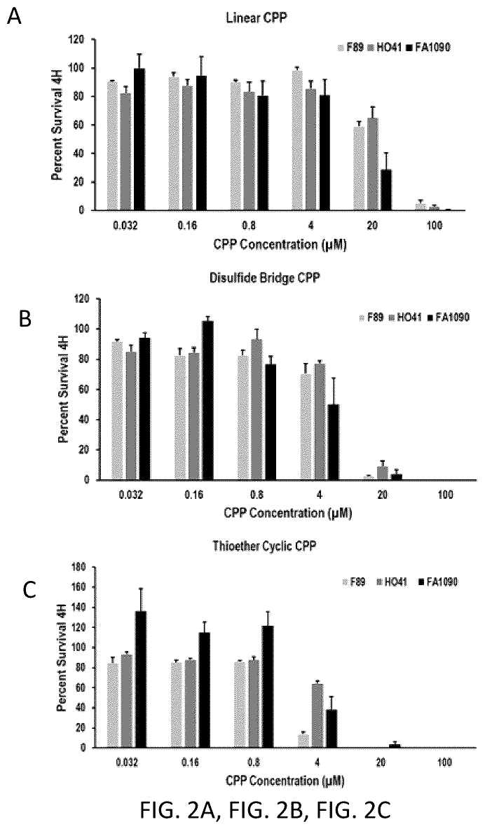

A bactericidal assay as a measure of potency of the cCPP compared to the linear CPP. Bacteria were incubated for 4 hours with increasing concentrations of each CPP and then plated for overnight growth and colony counting of the survivors. It was found that all three CPP were bactericidal for human challenge strain FA1090 and gonococcal MDR strains F89 and H041 (FIG. 2). However, the strains were significantly more susceptible to the cCPP compared to the linear CPP. Of the two cyclic structures, the thioether cCPP was consistently the most potent in bactericidal activity as illustrated in FIG. 2. This increased potency of the thioether cCPP compared to the disulfide cCPP could be due to the greater stability of the thiocther as a cCPP, increasing its membrane disruption or penetration of the membrane and/or bacteria. These data support the postulate that cyclization of the linear CPP can increase the efficacy of the cCPP for the bactericidal killing of gonococcal strains.

Additional measures of potency of the cCPP can include the effect on the induction of cytokines by human cells in response to gonococci, the effect on human cell invasion by gonococci, and cell cytotoxicity assays. In vivo studies can include the use of a female mouse infection model. To this end, female BALB/c mice are infected with MDR and human challenge strains of N. gonorrhoeae and treated with cCPP followed by culture of vaginal swab specimens and inflammatory cytokine measurements for 8 days thereby determining the efficacy of the cCPP in this established animal model of gonorrhea.

### B. Example 2

### 1. Background and Significance

i. Multidrug-Resistant Neisseria gonorrhoeae.

Infections due to N. gonorrhoeae are a major cause of morbidity with an estimated 1.6 million cases in the U.S. and 87 million cases worldwide annually. Within the VA Health Care System, cases of gonorrhea increased between 2013 and 2017 with the total number in that time period at 10,587. There is no vaccine to N. gonorrhoeae and a great need for new antibiotics due to the alarming rise in multidrug-resistant (MDR) strains, which is making emergence of untreatable gonococcal infections a real prospect. Surveys of antibiotic resistance in the population are the basis for treatment guidelines and when resistance to an antibiotic is more than 5%, its use is no longer recommended. Currently only ceftriaxone is recommended for first-line therapy, and clinical isolates resistant to this antibiotic have been reported in countries including Denmark, Canada, and Japan. Thus, there is a compelling need for new antimicrobials for gonococcal infections.

ii. N. gonorrhoeae Lipooligosaccharide and Inflammation.

The studies to date of N. gonorrhoeae lipooligosaccharide (LOS) and the human innate immune system have shown that LOS plays a major role in induction of innate immune inflammation. In particular, it was found that the degree of phosphocthanolaminylation and phosphorylation of the lipid A (LA) component is correlated with the potential of the LOS to induce inflammation through activation of both MyD88- and TRIF-dependent TLR4-MD-2 signaling pathways. These results are consistent with the concept that gonococci modulate the structure of LOS as a pathogen-associated molecular pattern molecule and that this modulation alters their interactions with the host in a manner that is likely to facilitate bacterial infection and survival. In addition to increasing the inflammatory potential of LOS as we have reported, other investigations have shown that substitution of the LA with phosphoethanolamine transferred by LptA also facilitates infection by increasing resistance to cationic anti-microbial peptides (AMP), inhibiting complement-mediated lysis of gonococci, and increasing resistance to the bactericidal activity of neutrophils. Further, the analysis of experimental infections in both mice and men with mixtures of wild-type N. gonorrhoeae and isogenic mutants that lacked LptA showed that LptA expression conferred a significant increase in the ability of the bacteria to survive in mammalian hosts. This application derives from past work on characterizing LOS as a primary inducer of cytokine-mediated inflammation, which led to the premise of this proposal that targeting LOS biosynthesis or outer membrane integrity can be an effective approach to combating N. gonorrhoeae infections.

iii. Cationic Cell-Penetrating Peptide Targeting of N. gonorrhoeae.

There is currently significant interest in cationic cell-penetrating peptides (CPP) and AMP as antibacterial agents due to the alarming rise of MDR Gram-negative bacteria. They were classically thought to kill microbes by depolarization and penetration of bacterial cell membranes and their selectivity for bacteria is thought to be due to structural differences between bacterial and mammalian membranes, the latter of which have net neutral zwitterionic outer leaflets comprised mainly of neutral lipids. However, recent studies indicate that the bactericidal activity of LL-37, which can be considered a prototypical AMP, is dependent on electrostatic binding of many positively-charged LL-37 molecules to the negatively-charged strands of chromosomal DNA and ribosomes, which impedes diffusive motion, rigidifies the cytoplasm, and causes microbial death. This provides a mechanistic explanation for the generally slow development of resistance to AMPs, which is an attractive attribute of their therapeutic potential.

The bactericidal potential of a 12 amino acid CPP was evaluated for MDR and human challenge strains of N. gonorrhoeae and it was found that the CPP penetrated the bacterial membrane and was bactericidal for all nine strains of gonococci tested with 95-100% killing of all gonococcal strains at a concentration of 100 μM. Importantly, there was no apparent resistance to the CPP that developed in surviving bacteria as susceptibility was the same in bacteria from colonies after a 4 h exposure to CPP and then retreated. Further, the CPP reduced cytokine induction and prevented bacterial cell invasion of cervical epithelial cells in the absence of measurable cell cytotoxicity. The results indicate that the continued development of CPPs as a treatment for gonococcal infections is warranted, especially as there is an urgent need for new therapeutic options for gonorrhea given the rise in MDR strains.

### 2. Significance

N. gonorrhoeae remains a major cause of sexually transmitted infections causing an estimated 87 million new cases worldwide. In the U.S. in 2021, 710,151 cases of gonorrhea were reported to the CDC and an estimated 1.6 million cases occurred, up 25% from 2017. N. gonorrhoeae has quickly developed resistance to all but one class of antibiotics and the emergence of MDR strains could result in untreatable gonorrhea due to resistance that is developing to the third-generation cephalosporins, ceftriaxone and cefixime, which are recommended first line treatments. As such, MDR N. gonorrhoeae is classified by CDC as an urgent public health threat that requires aggressive action.

If left untreated, or if treatment was ineffective, gonorrhea can lead to long-term health complications. In men, gonorrhea can result in symptomatic urethritis, epididymitis, and prostatitis. The most serious sequelae are suffered by infected women as gonococci ascend to the upper reproductive tract and cause pelvic inflammatory disease (PID) in 10-20% of women with infections. PID encompasses a wide range of inflammatory conditions and often leads to chronic pelvic pain, infertility, and ectopic pregnancy. Military and Veteran females, who incur a rate of gonorrhea that is higher than for civilian females and who suffer more frequent and serious complications than men, represent 15% of active duty military personnel and 2 million Veterans. Importantly, a growing number of studies have shown that gonococcal infection can facilitate the transmission of HIV. Further, control of gonococcal infection is widely recognized as increasingly challenging due to lack of any vaccine.

Within the VA Health Care System, cases of gonorrhea increased between 2013 and 2017 with the total number at 10,587. In general, although females comprise 10% of the VA patient population, they proportionally had increased positive test results for gonorrhea. In a study using national VA data to evaluate STI infection among 71,504 women Veterans who served in Operation Enduring Freedom/Operation Iraqi Freedom and sought care between 2001 and 2010, 0.17% had clinically documented gonorrhea. Similarly, a retrospective study among 996 women Veterans aged 51 years or younger who enrolled for care at two VA healthcare sites between 2000 and 2008 reported a 5% lifetime history of gonorrhea. For male Veterans versus non-Veterans, a recent study used nationally representative data from the National Survey of Family Growth 2011 to 2013 and reported that male Veterans were identified as being more likely than non-Veterans to have had gonorrhea in the past year (adjusted odds ratio=3.2).

Within the U.S. military, service members are a defined high-risk group for gonorrhea as noted by the U.S. Preventive Services Task Force as the high-risk environment of active military service is thought to increase risky behavior and, thus, the rate of sexually transmitted infections including gonorrhea. A total of 27,658 cases of gonorrhea were identified among active duty personnel during FY 2007 to 2015 (222.7 per 100,000 military compared with 179.3 per 100,000 non-military in 2018). Among active duty personnel deployed to Iraq and Afghanistan, women were found to have higher rates of gonorrhea than their male counterparts. In the U.S., a long-term study of active duty personnel at Fort Bragg, North Carolina showed that the adjusted incidence of gonorrhea among soldiers remained higher overall than comparable state and national rates among men and women during the period of analyses from 1985-1996.

Given the alarming rise of MDR strains of N. gonorrhoeae, additional drugs for treating gonorrhea are urgently needed. Recent data indicates that LOS and its biosynthesis as well as bacterial membrane stability are appropriate targets for the development of new therapeutic strategies for the treatment of gonococcal infections. These studies are focused on optimizing the structure of the CPP to increase their potency in multiple in vitro models of gonococcal infection. The CPP constructs can have bactericidal activity against MDR and human challenge gonococcal strains that can be predictive of in vivo efficacy in a female mouse model of gonorrhea.

The work described herein is innovative as it seeks to develop new drugs to treat N. gonorrhoeae infection. The data that N. gonorrhoeae is susceptible to CPP represents a major advance toward combating gonococcal infections with small molecule alternatives to or additions to the use of antibiotics. This current proposal derives from past work on innate immune responses to Neisserial LOS which demonstrated that LOS is the major inducer of the inflammatory cytokine response to gonococcal infection and that targeting LOS biosynthesis or outer membrane integrity can be an effective approach to combating antibiotic-resistant strains of N. gonorrhoeae. This study tests novel compounds from this CPP class of antimicrobials for in vitro and in vivo efficacy against N. gonorrhoeae.

### 3. Results

i. CPP is Bactericidal for N. gonorrhoeae.

The bactericidal activity of a 12 amino acid CPP was evaluated for human challenge and MDR strains of N. gonorrhoeae at concentrations from 0.032 to 100 μM based on efficacy data with several Gram-negative bacteria. The gonococci were incubated in cell culture media containing 10% FBS when assessing bacterial survival as serum has been shown to reduce the activity of AMPs likely due to binding to serum proteins such as albumin. The CPP was bactericidal for all nine strains of gonococci tested with 95-100% killing of all gonococcal strains at 100 μM. The strains demonstrated a dose-dependent susceptibility to the bactericidal activity of the CPP, and killing was observed down to a concentration as low as 0.032 μM.

ii. CPP Reduces Cytokine Induction by N. gonorrhoeae.

The effect of treatment of gonococci was investigated with CPP on inflammatory signaling of human THP-1 monocytes. Treatment with the CPP can modulate inflammatory signaling induced in THP-1 cells by killing the bacteria. To determine the effect on inflammatory signaling, THP-1 cells were incubated with bacteria at a multiplicity of infection (moi) of 1 in the presence of the CPP at 0, 4, 20, and 100 μM. It was found that levels of TNF-α in response to gonococcal infection of THP-1 cells by the MDR strains were significantly reduced by the CPP, indicating anti-inflammatory as well as a bactericidal activity for all strains tested.

iii. CPP Prevents Cell Invasion by N. gonorrhoeae in the Absence of Cell Cytotoxicity.

Because the CPP was bactericidal for N. gonorrhoeae and, furthermore, could penetrate the bacteria, it was postulated that treatment may prevent invasion of human epithelial cells. Therefore, the effect of the CPP on the invasion of a human cervical epithelial cell line, ME-180, was tested using the AMP LL-37 for comparison. FA1090 1-81-S2 gonococci at a moi of 5 were incubated with ME-180 cells in 96-well plates with vehicle only, LL-37 (20 μM), or the CPP (100 μM) for 4 h. Treatment with either agent completely inhibited invasion of the cells.

To determine whether the CPP was cytotoxic for THP-1 cells at concentrations that were bactericidal and reduced inflammatory signaling, the relative amount of lactate dehydrogenase (LDH) released from the cells was analyzed following 18 h treatment. Results showed that cell death was not significantly increased by treatment at either 100 or 200 μM concentrations which represent 5-fold and 10-fold more than the approximate MBC₅₀. By comparison, treatment of THP-1 cells with either 20 or 40 μM LL-37, which also represent 5-fold and 10-fold more than the approximate MBC₅₀ for LL-37, more than doubled the levels of cell death compared to the vehicle-only control (p<0.001).

These results demonstrate that the CCP is bactericidal for MDR and human challenge gonococcal strains. As a consequence of this killing activity, the CCP reduced gonococcal invasion of cells and induction of inflammatory cytokines in the absence of human cell cytotoxicity. Taken together, these results provide proof-of-concept data in support of further development of CPPs as a treatment for gonococcal infections, especially as there is an urgent need for new therapeutics for gonorrhea because of the rise in MDR strains.

### 4. Research Design and Methods

i. Optimization and in vitro testing of the CPP.
a. Modification and in vitro testing of the CPP.

### (A) Rationale

The urgent need for new treatments for N. gonorrhoeae is well recognized because the organism is increasingly MDR and the rate of infections is on the rise. There are promising results regarding bactericidal activity of a CPP, which has the structure (RXR)₄ (SEQ ID NO:4) where X is aminohexanoic acid, for human challenge and antibiotic-resistant strains of N. gonorrhoeae in vitro. The data revealed that the CPP killed 95-100% of all the strains at a 100 □M concentration and was not cytotoxic to human monocytic cells. Structural modifications of the CPP can be made to increase its (i) antimicrobial potency, (ii) selectivity for bacteria compared to human cells, and (iii) resistance to proteolysis. In addition, (iv) the effect of gonococcal efflux pumps can be assessed on the bactericidal activity of the CPP, and (v) its toxicity determined for human cells in new assay formats. In addition, a synthetic conjugate of CPP to the SCR 18-20 domain of human factor H (FH) can be produced based on the binding of FH SCR 18-20 to the porins PorB.1A and PorB.1B of sialylated gonococci, which can selectively target CPP to gonococci and thereby increase its potency. Further, the mutant of FH SCR 18-20 (D to G at position 1119 in domain 19) that binds to gonococci but does not lyse human erythrocytes can be generated.

### (B) Design

Most AMPs have several generalized features in common, amphipathicity, mean hydrophobicity and overall cationic charge. The cationic charge of AMPs is thought to be critical for selectivity because bacterial membranes are comprised of mainly negatively charged molecules such as phosphatidylglycerol, cardiolipid, lipoteichoic acids, or lipopolysaccharide, whereas the outer leaflet of the membranes of mammalian cells contains more neutral molecules, such as sphingomyelin, phosphatidylcholine, and sterols. Compared to lysine, the other positively charged amino acid, arginine has been found to be preferable in CPPs as a molecular transporter into cells because of its ability to form two hydrogen bonds with anionic cell surface molecules. This feature should increase its selectivity for bacterial compared to mammalian cells. In addition to increasing hydrophobicity and flexibility, the presence of 6-aminohexanoic acid residues in a peptide otherwise consisting only of arginine residues as in (RXR)₄ (SEQID NO: 4) is thought to increase serum and intracellular stability, possibly due to reduced susceptibility to proteolysis.

One strategy that can be used to increase the potency and selectivity of the CPP is to cyclize or “staple” it, which has been shown to be an approach that can constrain secondary structure, increase stability, and reduce the cytotoxicity of AMPs for mammalian cells. For example, a study focused on screening phage display peptide libraries showed that cyclization of two peptides decreased their minimum bactericidal concentration by 30-40-fold. Modified CPPs can be prepared by Anaspec. Initially a cyclized form of the CPP created through formation of disulfide bonds that are cleavable in vivo, by use of an (RXR)₄ peptide with two cysteines, can be tested. A construct with one cysteine at each end and a construct with one cysteine after (RXR)₂ (SEQ ID NO:5) and one after (RXR)₃ (SEQ ID NO: 6), i.e., CRXRRXRRXRRXRC (SEQ ID NO:1) and RXRRXRCRXRCRXR (SEQ ID NO: 3), respectively, can be tested.

To determine the relative specificity of the cCPPs, fluorescently-tagged peptides can be used, a strategy that we used to show uptake of FITC-labeled (RXR)₄ (SEQ ID NO:4) by N. gonorrhoeae. To address selectivity, FITC-labeled cCPPs produced by Anaspec can be incubated with N. gonorrhoeae alone and with mixed cultures of N. gonorrhoeae and mammalian cells. The gonococcal specificity of the FITC-labeled cCPP can be compared with and without conjugating it to SCR. The relative intensity of the intracellular fluorescence in the bacteria compared to the mammalian cells can be determined using confocal microscopy.

The relative bactericidal potency of the modified CPPs for the gonococcal strains can be determined, but clinical relevance of comparisons can be increased by also testing in the presence of human cells. This format has been proposed as the type needed to identify peptides that are able to achieve the cytoplasmic concentrations required to kill bacteria.

It has been shown that polycationic LL-37 is bactericidal for N. gonorrhoeae. Recent research in E. coli indicates that bactericidal activity of the polycationic LL-37 was due to its ability to form electrostatic networks in the cytoplasm as well as its ability to penetrate and permeabilize the bacterial membrane, which has been traditionally viewed as the mechanism of action of cationic AMPs. The data showed that high concentrations of LL-37 rigidified the cytoplasm by forming non-covalent bonds to chromosomal DNA and 70S-polysomes. A dual mechanism of action could help to explain why although evolutionarily very ancient, many types of bacteria are still sensitive to the bactericidal activity of AMPs. Thus, herein it is proposed to compare (i) interaction of the cCPPs with DNA by measuring the delay in migration caused by plasmid and chromosomal DNA in 1% agarose gel, and (ii) the ability of the cCPPs to disrupt bacterial membranes by measuring bacterial uptake of a fluorescent molecule.

A disadvantage of small peptides as drugs is a short circulating half-life, which typically is less than 60 min due primarily to proteolytic digestion. However, substitution of normal L-configuration amino acid residues with those in the D-configuration can greatly reduce susceptibility to proteolysis. For example, the octapeptide drug etelcalcetide, which was approved by FDA in 2017 for hyperparathyroidism, has 7 of its amino acid residues in the D-configuration and an effective half-life of 3-4 days. cCPPs can be tested in which L-arginine is substituted with D-arginine residues, which was shown to reduce proteolysis of arginine-rich CPP morpholino conjugates. Furthermore, as mentioned above, cyclization can increase the circulating half-life of peptides. In vitro assays can be used to model in vivo half-life by measuring proteolysis rates in 10 and 50% human serum and in liver lysosomal preparations with RP-HPLC analysis.

The cCPP can be conjugated to the SCR 18-20 of FH to increase the specificity of the cCPP for sialylated gonococci. A cCPP can be produced with an N-terminal cysteine and form a disulfide bond to the C-terminal cysteine on the recombinant SCR 18-20 that will specifically target gonococci. The interaction in vivo of the conjugate with reduced glutathione and cysteine present in mucosal membranes is expected to cleave the disulfide over time, resulting in increased local concentration of free cCPP at the membrane of gonococci and overall potency. Cysteine linkages to produce disulfide conjugation have been one of the most common methods used to prepare antibody drug conjugates, which are a major new class of therapeutic agents.

One of the disadvantages of some AMPs such as LL-37 and polymyxin B is a relatively low threshold for toxicity or adverse side effects. In addition to testing the modified CPPs for cytotoxicity to activated human THP-1 monocytes, the minimum hemolytic concentration (MHC) can be determined, which is the minimum concentration that causes hemolysis of red blood cells, and cytotoxicity for other non-adherent and adherent human cell types can be determined.

b. Methods

### (A) Bactericidal and Cytokine Assays in the Presence of Human Monocytic, Endothelial, and Epithelial Cells.

Bactericidal assays of the cCPPs can be performed with gonococci alone. Bactericidal assays can also be performed in the presence of (i) activated human THP-1 monocytic cells (ATCC), (ii) immortalized endocervical cells (End1/E6E7; ATCC), (iii) immortalized cervical cells (ME-180; ATCC), (iv) primary urethral epithelial cells (Cell Applications, Inc.), and (v) primary cervical epithelial cells (Cell Applications, Inc.) to increase the potential clinical relevance of the findings. Ideally testing is performed in the presence of cells that are of both male and female origin and of a variety of cell types. For most in vitro testing five prototypical strains can be utilized that have been used previously: two human challenge strains, FA1090 1-81-S2, MS11mkC, and three MDR strains, F89 (WHO Y), H041 (WHO X) and WHO L. The use of strain H041 can be particularly informative as this strain has been shown previously to rely predominantly on the MtrCDE efflux pump to achieve its high-level resistance to ceftriaxone. To determine the effect of LOS sialylation, experiments will be performed with and without cytidine-5′-monophospho-N-acetylneuraminic acid (CMP-NANA) by adding CMP-NANA to a final concentration of 10 μg/ml to the growth media. To determine specificity for Gram negative bacteria, a strain of Gram positive S. aureus (ATCC #13709) can be propagated in nutrient agar and used in bactericidal assays.

THP-1 cells can be cultured in RPMI 1640 with 10% FBS (growth medium) and activated by incubation with 10 ng/mL phorbol myristate acetate (Sigma-Aldrich) in growth medium for 18 h. End1/E6E7 cells will be maintained in Keratinocyte Serum-Free medium (Thermo Fisher) supplemented with 0.05 mg/ml bovine pancreatic extract, 0.1 ng/ml human epidermal growth factor, and 0.44 μg/ml CaCl₂). Primary urethral and cervical epithelial cells will be cultured in Epi Growth Medium with Growth Supplementation (Cell Applications). Human ME-180 cervical epithelial cells will be cultured in McCoys 5A growth media with 10% FBS (Gibco) in 5% CO₂ at 37° C. When comparing results between the various cell lines, the presence or absence of FBS in each cell type will be factored into the data analysis to determine whether FBS may affect the activity of the cCPPs through protein binding.

The human cells can be plated in 48-well plates (2×10⁵ cells/well) and when confluent challenged with N. gonorrhoeae at a moi of 10. N. gonorrhoeae can be grown overnight on GC agar with 1% Isovitalex in the presence or absence of 10 μg/ml CMP-NANA and mid-log phase bacteria can be diluted in media and plated into wells with human cells. Vehicle controls containing 1% DMSO will be included. Bactericidal activity of the cCPPs will be assessed in triplicate from growth of bacteria from 10 μl aliquots of serial dilutions of both supernatants and lysed cells from wells. As a control to assess the bactericidal killing of extracellular bacteria in the absence of intracellular bacterial uptake, cytochalasin D (3 μM; Sigma-Aldrich) can be used to block invasion. For analysis of the supernatants, aliquots will be obtained after shaking in a CO₂ incubator at 37° C. for 2 hours. After shaking for 2 more hours, aliquots of a stock solution of gentamicin in water will be added to each well to yield a concentration of 100 μg per ml and plates can be incubated for an additional 2 h. After washing wells twice with PBS, 1% saponin (Sigma-Aldrich) in PBS will be added and plates will be incubated for 20 min. Finally, 10 μl aliquots of serial dilutions from each well can be plated on GC agar plates to quantify viable intracellular bacteria by counting colonies.

From these data, the minimum bactericidal concentration (MBC), which will be defined as the lowest concentration of CPP that results in at least 99.9% killing of the initial inocula, can be determined. Improved potency can be defined as statistically significant decreases in the MBC. For the cCPPs, MBC of >100 μM can be considered poor, 20-100 μM medium, and <20 μM good. The assays can then be repeated using a concentration of cCPP that is 50% MBC to determine whether multiple bacterial passages result in an increase in resistance to the agents.

For cytokine analysis, supernatants from representative infected cultures incubated without shaking for 0, 4, and 23 h, can be harvested, centrifuged, and stored at −80° C. for analysis using a BioPlex Human Cytokine Assay (Bio-Rad). Based on prior N. gonorrhoeae incubation with urethral and endocervical epithelial cells, increases in expression of IL-6, IL-8 and TNFα from bacteria or LOS exposures are expected. In addition, LOS will be quantified using LOS MAb 2C7 in an ELISA to determine LOS levels in proteinase K-treated samples as a measure of the PAMP burden during infection.

### (B) Bactericidal Assays Revealing Susceptibility to Efflux Pumps.

To determine the susceptibility of the CPPs and the CPP-FH conjugate to efflux pumps, the bactericidal activity can be compared in wild type and mtrC, mtrD, mtrE, macA, macB, and norM mutant strains and in strains with IPTG-inducible promoters of the efflux genes to study the effect of different levels of gene expression. Bactericidal activity can also be assessed by incubation with gonococci in the presence of the PABN efflux pump inhibitor.

(C) Analysis of Secondary Structure of cCPP.

The secondary structure of cCPPs can be analyzed by circular dichroism (CD) spectroscopy on a the Jasco-J-714 CD spectrometer. CD spectra will be obtained in PBS at pH 7.4 and in PBS with 25 mM SDS micelles, 50% trifluoroethanol (TFE) or 10 μM gonococcal LOS. Spectra can be recorded from 200-250 nm to determine fractional helicity using two independent measures of mean residue molar ellipticity at 222 nm.

(D) Preparation of cCPP-FH SCR 18-20 Conjugate.

Disulfide bond formation can be used to conjugate the cCPP to the SCR domains 18-20 of the FH protein that can have an N-terminal cysteine, which should be sterically favored for reaction with the peptide. Established protocols can be tested to identify an optimal condition. The concentration of the cCPP can be kept low relative to that of the protein to prevent formation of intramolecular cCPP disulfide bonds. The cCPP-SCR 18-20 FH product will be purified with reversed-phase HPLC on a C4 column. The formation of intramolecular disulfide bonds can be tested using potassium ferricyanide oxidation as follows. A solution of the cCPP (0.1 mg/ml) and the SCR 18-20 (1.0 mg/ml) will be dissolved in PBS and the solution will be titrated with 0.01 M aqueous K3Fe (CN)6 solution until it is slightly yellow. The pH can be adjusted to 5 with 50% aqueous acetic acid and the oxidant removed with an AG-3 anion-exchange resin. The conjugate can be analyzed by MS and its ability to bind to N. gonorrhoeae sialylated with CMP-NANA will be determined using a whole bacteria ELISA and Quidel factor H MAb A229 that binds to the SCR domain 18-20 of FH. Alternatively, oxidation can be used by DMSO under slightly acidic or slightly basic conditions and monitor the progress of the reaction using HPLC.

### (E) DNA Binding Assays.

Because the nature and conformation of DNA may impact how cCCPs interact with DNA, a modified gel retardation experiment can be used to compare the DNA binding ability of the cCPPs. Briefly, a fixed amount of pBR322 plasmid DNA (100 ng), (supercoiled form, linearized form generated by digestion with a restriction enzyme, and relaxed form generated by incubating plasmid DNA with a DNA nicking enzyme available from New England Biolabs) or chromosomal DNA (100 ng) can be mixed with various concentrations of cCPPs in sample buffer (10 mM Tris-HCl, 5% glucose, 50 μg/ml BSA, 1 mM EDTA and 20 mM KCl) and incubated at 37° C. for an hour. The samples can be analyzed in 1% agarose gel electrophoresis in 0.5% TAE buffer with DNA detection using a UV illuminator to determine the lowest concentration of CPP that alters or completely inhibits the migration of the DNA. If interactions with DNA are demonstrated, one could then investigate alterations of transcription in the presence of cCPPs.

### (F) Protein Binding Assays.

The well-established method of rapid equilibrium dialysis (RED) can be used to determine the extent to which the cCPPs bind plasma proteins. Human plasma can be spiked with test compounds and 200 μL added to inner chambers of a 48-well RED (ThermoFisher) plate with 8 kDa MWCO inserts. Isotonic phosphate buffer (200 μL) can be added to outer chambers of the plate that can then be incubated on an orbital shaker at 300 RPM at 37° C. for 1-4 h. Aliquots (100 μL) of samples can be added to microcentrifuge tubes and a corresponding amount of buffer (100 μL) will be added to the plasma sample, and plasma (100 μL) will be added to the buffer sample. Precipitation buffer (300 μL of cold 90:10 acetonitrile/water with 0.1% formic acid with internal standard) can be added and the plate vortexed and incubated on ice for 30 min to precipitate protein and release compound. After centrifugation the supernatant will analyzed by LC-MS/MS. Values will be: % Frec=(concentration buffer/concentration plasma)×100. % Bound=100%−% Free. The in vivo concentration of bioavailable drug will be that unbound by plasma proteins.

### (G) Membrane Permeabilization and Bactericidal Assays in the Presence of Polymyxin B Nonapeptide (PMBN).

PMBN is a cyclic form of polymyxin B that lacks its bactericidal activity but retains the ability to permeabilize bacterial membranes. During bactericidal assays wells with 8 μg/ml PMBN can be included to determine whether increasing membrane permeabilization can increase bacterial sensitivity to the cCPP. Furthermore, the ability of the cCPPs and cCPP-FH conjugate to permeabilize bacterial membranes can be directly quantified. The penetrability of the outer membrane can be determined based on increases in fluorescence from bacterial uptake of 1-N-phenylnaphthylamine (NPN). NPN is a hydrophobic molecule that fluoresces in hydrophobic environments such as lipid membranes but only fluoresces weakly in aqueous environments. Gonococci growing on GC agar plates can be harvested, washed, diluted and plated in PBS in 96-well black microplates to which NPN in 95% ethanol can be added. Thereafter, the cCPPs, cCPP-FH conjugate, or vehicle can be added to wells and the effect of each can be determined using excitation and emission wavelengths of 350 and 420 nm, respectively.

### (H) Hemolysis Assays.

In vitro hemolysis of red blood cells can be measured as a surrogate for nonspecific mammalian cell toxicity. Hemolytic assays can be performed to determine cytotoxicity of the cCPPs and cCPP-FH conjugate as previously described (66). The assays can be performed using sheep red blood cells (SRBCs; Rockland, Limerick, PA). The sRBCs can be washed and suspended in PBS and dispensed into 96-well plates. Aliquots of solutions with the cCPP, cCPP-FH conjugate or a 1% DMSO vehicle control can be added to each well and after incubation for 1 h at 37° C., samples will be centrifuged and supernatants (100 μL) can be transferred into a 96-well plate. sRBCs incubated with PBS or 0.1% Triton X-100 can be negative and positive controls, respectively, and treatments will be performed in triplicate. Percentage of hemolysis can be derived from the following equation: % Hemolysis=[(Abs405 nm in the peptide solution-Abs 405 nm in PBS)/(Abs 405 nm in 0.1% Triton X-100−Abs 405 nm in PBS)]×100. Values of >25% hemolysis will be defined as poor, 10-25% medium, and <10% good.

### (I) Cytotoxicity for Human Monocytic, Endothelial, and Epithelial Cells.

These assays can be performed with activated human THP-1 monocytic cells, immortalized End1/E6E7 human endocervical cells, immortalized human urethral epithelial cells, and human ME-180 cervical epithelial cells that can be cultured. Inhibitors can be assayed that show activity in bactericidal assays and testing can be performed at the concentration at which 50% of the bacteria are killed in the bactericidal assay, and at 2.5- and 5-fold higher concentrations. Release of LDH from dying cells into cell culture after exposure to the cCPPs or the cCPP-FH conjugate can be analyzed to determine cytotoxicity. Briefly, cells can be plated (1×10⁴ per well) in Opti-MEM media (Gibco) with 1% FBS in 96-well plates. Once the cells have adhered to wells, aliquots of stock solutions of the cCPP will be added. Control cells will be treated with vehicle only. Total reaction volumes will be 100 μL per well. Plates will be incubated for 37° C. in 5% CO2 for 18 h and then the levels of LDH in supernatants can be quantified using a kit (Roche, Mannheim, DE) with detection on a Thermomax plate reader at 490 nm. Results can be reported as the percentage of LDH released from treated cells relative to LDH released from vehicle-only control cells. Values of >10% cytotoxicity will be considered poor, 2-10% medium, and <2% good.

(J) Uptake of cCPP by Bacteria and Human Cells.

N. gonorrhoeae and human cells can be incubated with FITC-labeled cCPPs (10 μM) and the DNA stain, 4′,6-diamidino-2-phenylindole, in GC broth or cell culture media for 1 h to determine the relative uptake of the cCPPs. After trypsinizing the human cells, cells and bacteria can be washed with PBS three times and then aliquots containing cells or bacteria will be air-dried onto a glass slide. Analysis of uptake can be by confocal microscopy.

(K) cCPP Proteolysis in Serum and Lysosomal Preparations.

Proteolysis can be tested in both serum and liver lysosomal preparations as the enzymes active in these compartments are expected to vary. Human serum can be obtained commercially from Thermo Fisher Scientific and proteolysis of the cCPPs can be determined in 10 and 50% serum with PBS. Peptides in solution at the same concentration, but without serum can be used as controls. Peptides can be incubated at 37° C. and samples can be taken after 0, 24, 48, 72, and 96 h. Reactions can be stopped by adding 5% (vol/vol) perchloric acid. After centrifugation, the concentration of intact peptide in the supernatant can be determined by reverse-phase (RP) HPLC. Each sample can be analyzed twice and MS/MS analyses can be performed to confirm the results.

To prepare liver lysosomal preparation, livers can be collected from five mice after euthanasia. The livers can be homogenized in two volumes of ice-cold 0.3 M sucrose with 10 strokes at 15×g. After dilution with three volumes of 0.3 M sucrose, the preparation can be centrifuged at 700×g for 10 min. Then the supernatant can be centrifuged at 10,000×g for 10 min to sediment the lysosomal-mitochondrial fraction, which can be homogenized in 10 ml of 0.3M sucrose with 1 mM CaCl2. After incubation of the homogenate at 37° C. for 5 min, 10 ml of 50% Percoll solution can be added, and the homogenate can be centrifuged again at 10,000×g for 10 min to obtain the brown pellet that can be the lysosomal fraction. As a control enzymatic activity of the preparation can be confirmed with BSA as the substrate.

To analyze the proteolysis in the lysosomal preparation, cCPPs can be dissolved in 0.1 M acetate buffer (pH 3.5 or 5.0) and aliquots of the lysosomal fraction can be added for a final 1/50 dilution. cCPPs can be incubated at RT and samples can be taken at 30 sec, 60, 120 and 180 minutes. Enzymatic reactions can be stopped by adding 5% (vol/vol) perchloric acid. After centrifugation at 3,500×g for 5 min at −4° C. the concentration of the intact peptide tohu be determined by RP-HPLC. Peptides in acetate buffer without the lysosomal fraction can serve as the negative control.

### (L) Statistical Methods of Data Analysis

The experimental design includes sufficient replicates to ensure the reproducibility of our results. The rigor of the results can be demonstrated by the following statistical analyses. Differences before and after experimental modification can be analyzed with the Wilcoxon signed rank test for comparison of matched samples. Comparison of two different groups can be analyzed with the Mann-Whitney U test. Comparison of three or more groups can use Tukey's multiple comparison statistic. Where a normal distribution of data can be determined by analysis of skew and kurtosis, parametric tests can be performed.

c. Expected Results

The cyclized form of the CPP can have more stability and/or more selectivity for bacterial cells than the linear CPPs, and the D-amino acid cCPP should be more stable and have undiminished activity compared to the analogous L-amino acid cCPP.

The cCPP-FH conjugate can be more efficacious than CPP due to bacterial localization.

An alternative method to assess damage to the bacterial cell membrane can be flow cytometry for analysis of bacterial uptake of PI from a solution of 2 mg/ml.

ii. Determine the In Vivo Efficacy of the CPP in Female Mouse Model.
a. Rationale

Relating in vitro antimicrobial drug data to the potential for human therapeutic benefit has been challenging due to the lack of suitable animal models for gonorrhea. Over the past decade however, a female mouse model of gonococcal genital tract infection has been increasingly used for preliminary evaluation of candidate antimicrobials for treatment of gonorrhea. A model has been developed using estradiol-treated female mice in which the animals can be reliably infected providing an experimentally modifiable means of studying the impact of antimicrobials on gonococcal pathogenesis and the course of experimental infection. It was found that CPP was bactericidal for MDR and human challenge strains of gonococci and reduced cytokine induction without apparent cell cytotoxicity. In these experiments, the plan is to test two molecules, the lead candidate cCPP and a conjugate of it with FH SCR 18-20 that can target gonococci, in the well-characterized female mouse model of gonorrhea. The hypothesis to be tested is that these antimicrobial agents can cause a therapeutic reduction in both the duration and burden of infection. This study can be the first of its kind to report that cCPP is an effective antimicrobial agent against MDR strains of N. gonorrhoeae. To this end, mice will be infected with MDR and human challenge strains of N. gonorrhoeae and treated them with the lead candidate cCPP and the fusion of FH SCR 18-20 with the cCPP followed by culture of vaginal swab specimens and inflammatory cytokine measurements for 8 days thereby determining the efficacy of these antimicrobials in this established animal model of gonorrhea.

b. Design

In in these experiments, the optimal cCPP and a cCPP conjugated to FH SCR 18-20 will be studied in vivo. Pharmacokinetic (Pk) analyses, safety studies including maximum tolerated dose (MTD) and cardiovascular (CV) assays can be performed, and efficacy will be determined in a well-established female mouse model of gonorrhea. These are all critical steps in translating the findings into potential clinical candidates. Statistically significant differences of potency and efficacy of these agents can be determined in duration of infection, total bacterial burden, and levels of inflammatory cytokine markers in the mouse model.

c. Methods

### (A) Bacterial Strains.

N. gonorrhoeae strain FA1090 has been widely used in human challenge studies and in one of these studies the 1-81-S2 strain was isolated. F89, classified as WHO Y, was isolated in France from a patient with a urethral infection in 2010 and was found to have resistance to ceftriaxone and most other antibiotics tested. H041, classified as WHO X, is a highly ceftriaxone-resistant isolate from the throat of a patient in Japan in 2009 that is considered an MDR strain due to its extensive resistance to antimicrobials. All three of these strains have been previously shown by consultant Dr. Jerse to establish infection in the female mouse model of gonorrhea.

(A) Pharmacokinetic (Pk) Analysis of cCPP in Plasma.

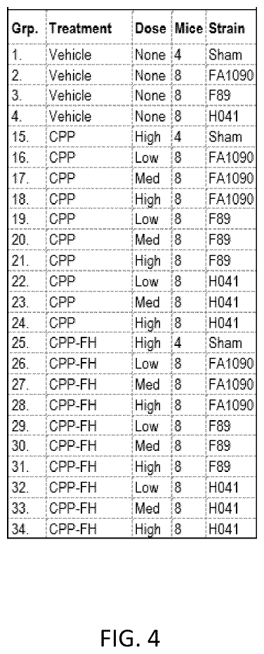

cCPP plasma concentrations can be determined by liquid chromatography (LC) with tandem mass spectrometry (MS/MS) using an Accela HPLC and LTQ XL ion trap mass spectrometer. The data can be used to generate a concentration-time curve to determine the area under the curve (AUC), volume of distribution (VD), and the elimination half-life (t1/2). The antibiotic and estrogen treatment protocol described in the female mouse infection model below can be followed for plasma sample collection from uninfected female BALB/c mice for Pk analysis. As shown in FIG. 4, blood samples can be collected by terminal cardiac puncture from 4 deeply anesthetized mice at each time point: 0.08, 0.25, 0.5, 1, 2, 4, 8, 12, and 36 h after administration of the cCPP and processed to serum for cCPP. Although the plasma cCPP Pk can be obtained using plasma from uninfected mice, the presence of a localized vaginal gonococcal infection would not be expected to dramatically impact the Pk dynamics of the agent.

The cCPP can be analyzed using known methods. Serum samples can be used for multiple reaction monitoring (MRM) MS analysis in which the parent mass of the analyte is selected for MS/MS fragmentation and a single fragment ion is monitored. Samples (100 μl) will be spiked with an internal standard (human insulin, 2.3 nmol/ml), combined with a 50 mM acetic acid solution, loaded on a C18 column and eluted with 40% acetonitrile/50 mM acetic acid. The solution can be dried under vacuum, resuspended in 20 μl 0.1% formic acid and 10 μl will be injected onto the column with a linear gradient of water/acetonitrile with 0.1% formic acid and analysis of the effluent via positive-ion electrospray ionization (ESI) MS/MS with MRM.

### (B) MTD Assay.

In conjunction with the Pk analyses, MTD assays can be performed. Female mice can be treated with antibiotics and estrogen as described in the female mouse infection model below. For each peptide, groups of 4 mice can be injected i.p. with a concentration of cCPP that is κ× the highest dose administered in the infection and Pk models or with a vehicle control. All mice can be weighed prior to and at dosing and observed twice daily through day

### 7. Weight Loss and Clinical Score Based on Activity, Appearance and Body Condition can be the Endpoint Criteria

N. gonorrhoeae

### (C) In Vivo Efficacy Testing in the Female Mouse Model.

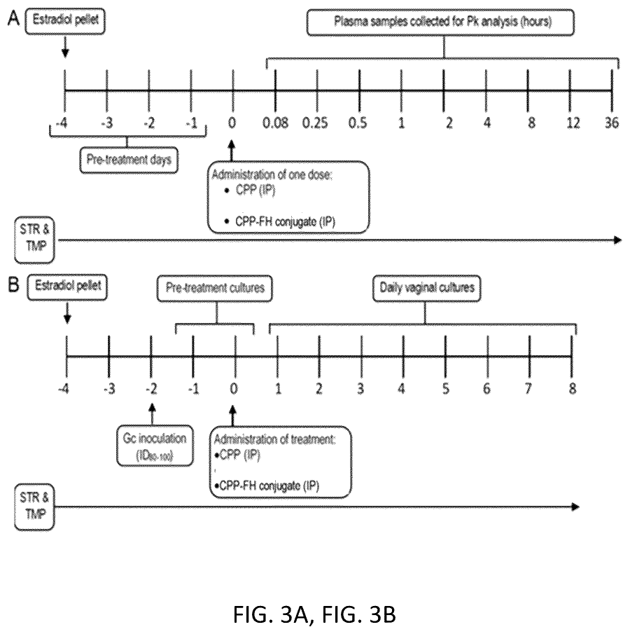

Female BALB/c mice (6-7 weeks old; Charles River Laboratories) in the diestrus or anestrus stages of the estrous cycle as determined by cytological examination of vaginal smears will be implanted with a 5 mg, 21-day-slow-release 17ß-estradiol pellet (Innovative Research of America) and treated with antibiotics to promote long-term gonococcal infection using the standard infection protocol. As shown in FIG. 3, the antibiotics can be used to suppress commensal flora overgrowth and will include streptomycin (24 mg/ml; Sigma) administered via intraperitoneal (i.p.) injection twice daily on days-4 through-1, and trimethoprim (MP Biologicals) given in the drinking water (0.4 g/liter) through day-1. Both antibiotics at a concentration of 5 g/liter will be provided in the drinking water from day 0 through the remainder of the study duration. Mice will be inoculated with N. gonorrhoeae on day-2 by first rinsing the vagina with 30 μl of 50 mM HEPES (pH 7.4) followed by inoculation with 20 μl of a PBS suspension containing 106 CFU FA1090 or 104 CFU H041 or F89 gonococci. This inoculum has been shown previously to be the lowest that will infect 80-100% of mice for at least 10 days for each strain. Controls can be inoculated intravaginally with diluent. For testing the cCPP-FH conjugate, 10 μg of CMP-Neu5Ac can be administered locally on a daily basis to ensure LOS substitution with Neu5Ac as mice express both CMP-Neu5Ac and CMP-Neu5Gc unlike humans who express only CMP-Neu5Ac.

Following collection of specimens for culture on days-2 to 0 after inoculation to confirm infection, the cCPP will be administered intraperitoneally to mice as a single dose (0.2 ml, i.p.) of from 1 mg/kg up to 125 mg/kg. PBS administered via the same i.p. route will serve as the negative control. As a positive control, gentamicin will be administered i.p. (0.2 ml) to 4 untreated mice infected with strain H041 once per day for 5 days. Gonococci can be harvested from the vaginal mucus of all mice using a rayon swab moistened with PBS that will be suspended in 1 ml of GC broth followed by culturing of undiluted and diluted samples on GC-VCNTS agar. The infection can be monitored by culture of vaginal swab specimens for 8 days following treatment. Mice will be considered to have cleared the infection when cultures of vaginal swab specimens are negative for colony counts for three or more consecutive days. In addition to bacterial culture, levels of inflammatory cytokines can be determined. Vaginal washes from mice used in the infection experiments will be collected on days 1, 3, 5, and 7 by gently pipetting 50 μl of PBS in and out of the vagina 20 times. The lavage fluid will then be centrifuged at 13,000×g for 3 min. The supernatant can be frozen immediately and stored at −80° C. for further analysis. Levels of IL-1β, TNF-α, CXCL1, and MIP-2 proteins can be measured using a BioRad multiplex system. Endpoints for the infection experiments can be duration of infection, bacterial burden, and levels of inflammatory cytokine markers. The treatment groups and numbers of mice are summarized in FIG. 4.

### (D) Statistical Methods of Data Analysis

The experimental design includes sufficient replicates to ensure the reproducibility of our results. The rigor of the results can be demonstrated by the following statistical analyses. Differences in colonization load in the cCPP treatment groups compared to the control group can be assessed by a repeated-measures two-way analysis of variance (ANOVA) using Bonferroni's post hoc analysis for multiple pairwise comparisons. Differences in the duration of colonization can be assessed using a Kaplan-Meier survivorship curve and the log-rank (Mantel-Cox) test. Comparison of cytokine levels can use Tukey's multiple comparison statistic.

d. Possible Outcomes

The estradiol-treated mouse model of N. gonorrhoeae genital tract infection has proved invaluable for addressing questions regarding gonococcal infection in vivo. In these experiments, the results can demonstrate the translational potential of the CPP antimicrobial for targeting gonococcal infection and justify further research towards clinical testing.

Depending on the results, it is possible that alternative or multiple dosages of the cCPP will need to be tested and, if so, additional groups of mice may be needed. In this model, the possibility exists that commensal bacteria may produce a potential inhibitory environment if the antibiotics do not fully suppress the overgrowth of inhibitory commensal flora that occurs under the influence of estrogen. To test for this, heart infusion agar can be inoculated with a portion of the vaginal swab to screen for potentially inhibitory commensal bacteria such as P. aeruginosa, Proteus spp., or other members of the Enterobacteriaceae family that might confound clearance data. As gonococci can colonize diverse host sites including male versus female, lower versus upper genital infection, and genital versus extragenital sites, one pitfall of the female mouse model is that it is not known whether results from the model are predictive of bacterial clearance kinetics in the urethra, rectal or pharyngeal mucosal sites of infection. One approach to gain information on treatment of gonococcal infections at these anatomical sites can be to use primary urethral, rectal and pharyngeal human cells in an in vitro infection model. As described above, primary urethral cells can be used in the in vitro cell-based assays.

### C. Example 3: Cyclic Cell-Penetrating Peptides as Therapeutics for Neisseria Gonorrhoeae Infection

Neisseria gonorrhoeae has quickly developed resistance to antibiotics and the rapid emergence of multidrug-resistant (MDR) strains could result in untreatable gonorrhea. A linear 12 amino acid cell-penetrating peptide (CPP) was bactericidal for MDR strains of N. gonorrhoeae was previously designed.

A cCPP was synthesized using head-to-tail macrocyclization and bactericidal assays were performed as a measure of potency. Survival of two MDR strains, F89 (WHO Y), H041 (WHO X), and one human challenge strain, FA1090 1-81-S2, was determined after treatment with increasing concentrations of the cCPP and linear CPP for 4 h. To assess anti-inflammatory activity, the expression of TNF-α by THP-1 monocytic cells infected with gonococci in the presence of increasing concentrations of CPP was quantified. To determine CPP activity in a more physiological environment, bactericidal assays were performed in the presence of THP-1 monocytic, End1/E6E7 endocervical cells and ME-180 cervical cells in cell culture media containing 10% FBS for 4 h. Bactericidal activity of the CPP for three commensal strains, N. lactamica, N. flavescens, and N. subflava and for gram-positive Staphylococcus aureus was tested.

The linear and cCPP were bactericidal for all three gonococcal strains, however, the strains were significantly more susceptible to cCPP compared to linear CPP in the concentration range of 4-100 μM. Similarly, in the presence of THP-1, End1/E6E7 cells and ME-180 cells, the cCPP was significantly more bactericidal than the linear CPP. The levels of TNF-□ secreted by THP-1 cells infected by gonococci were reduced to a greater degree by treatment with the cCPP compared to the linear CPP. Commensal strains were more susceptible to the cCPP, whereas S. aureus was resistant with an MIC of >100 μM.

The data shows that cyclization of CPP can increase potency for the bactericidal killing of gonococci. This increased potency may be due to the more rigid conformation of the cyclic form, which could increase its penetration of bacteria or its affinity for DNA intracellularly.

### D. Example 4

### 1. Cyclic CPPs are More Potent in Bactericidal Assays with Human Challenge and MDR Strains of N. gonorrhoeae

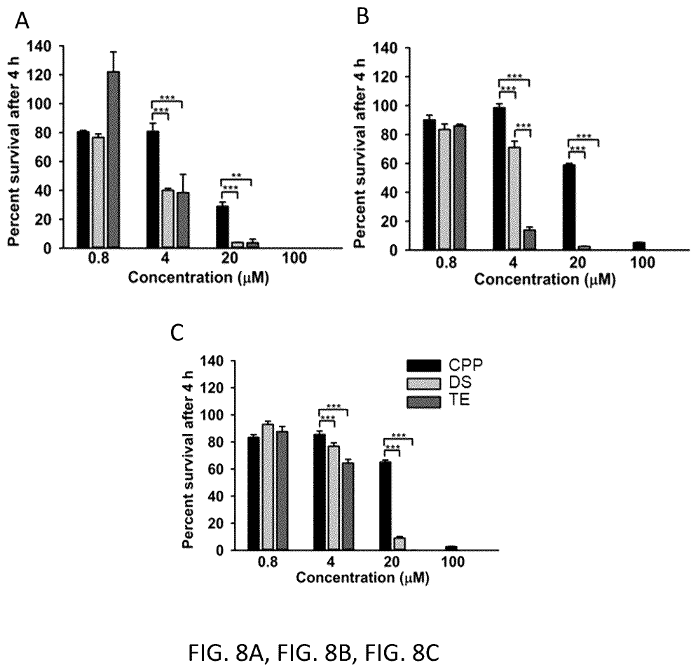

Four-hour treatment with the linear CPP, DS (disulfide) cyclized CPP, and the TE (thioether) cyclized CPP was bactericidal for the gonococcal strains (FIG. 8A-C). The gonococci were significantly more sensitive to both cyclized CPPs (DS cyclized CPP, and the TE cyclized CPP) compared to the linear CPP at both 4 and 20□M. There was complete killing of the gonococcal MDR strain F89 and H041 isolates and >90% killing of FA1090 by the TE cyclized CPP at 20 □M. At 4 □M, the F89 bacteria were significantly more, and the H041 bacteria tended to be more sensitive to the TE cyclized CPP compared to the DS cyclized CPP.

### 2. TE Cyclized CPP is not Cytotoxic for Human Cells

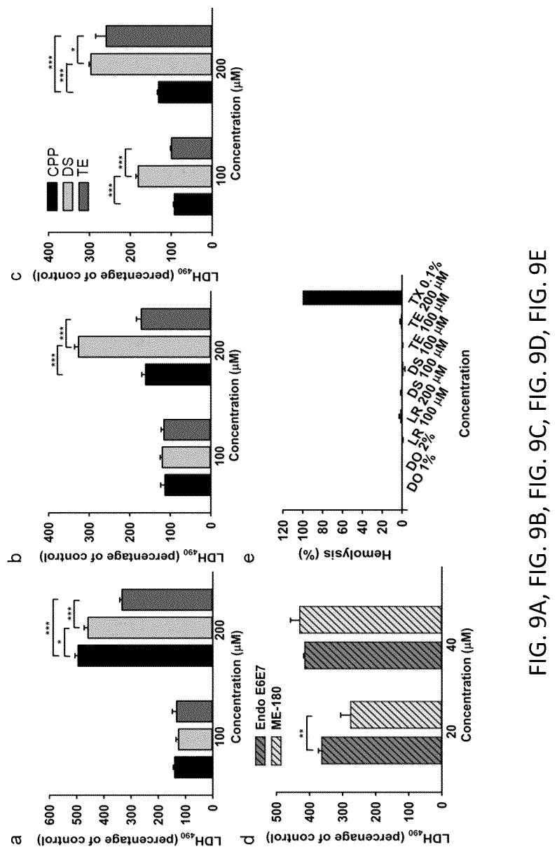

To determine whether the CPPs were cytotoxic for human cells, the relative amount of LDH released from immortalized End1/E6E7 endocervical cells, ME 180 cervical cells and THP-1 monocytic cells was measured following 18 h treatment (FIG. 9A-C). Results showed that at the 100 □M concentration, which represents 10-fold more than the approximate MBC₅₀, cell death was significantly increased only in THP-1 cells treated with the DS cyclized CPP. At the 200 μM concentration, the TE cyclized CPP was less cytotoxic compared to the DS cyclized CPP for all three cell types. At the 200 μM concentration, the TE cyclized CPP was also less cytotoxic compared to the linear CPP for the End1/E6E7 cervical cells but more cytotoxic for the THP-1 cells. The cytotoxicity of the endogenous AMP LL-37 was analyzed as a comparator. Treatment of the End1/E6E7 or ME-180 cells with LL-37 at the 20 □M concentration (FIG. 9D) more than doubled the LDH levels.

### 3. CPPs do not Cause Hemolysis

In vitro assessment of hemolysis is important in determining the potential cytotoxicity of any material or drug that would be in contact with blood. Various protocols for determining hemolytic potential have been reported. A procedure recommended for analysis of the hemolytic potential of drug substances for RBCs using 0.1% Triton as the positive control and a 1 h incubation period was performed. The results (FIG. 9E) showed that there was no hemolysis from the linear, DS- or TE-cyclized CPPs at either the 100 or 200 M level.

### 4. TE-Cyclized CPP is More Bactericidal for Sialylated Gonococci than the Linear CPP

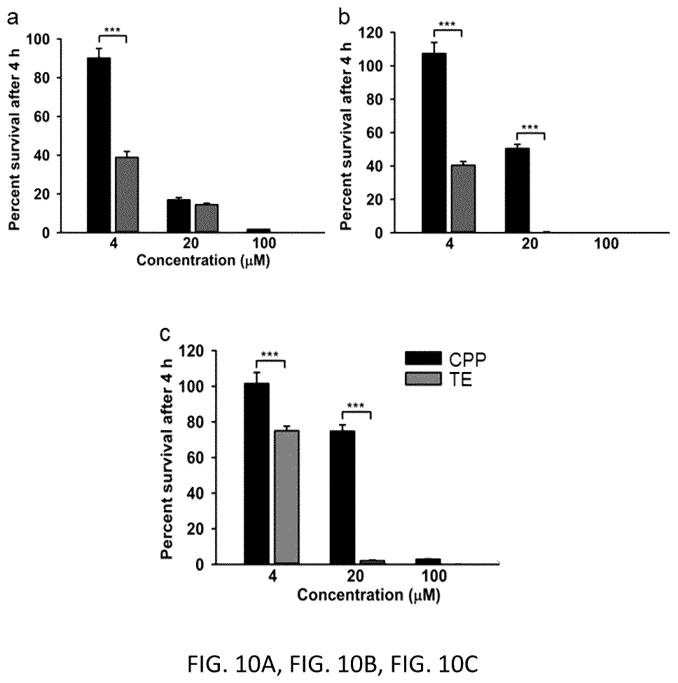

Subsequent efforts were focused on comparing the linear CPP only with the TE-cyclized CPP which had more potent bactericidal activity (FIG. 8) and less cytotoxicity than the DS-cyclized CPP (FIG. 9). Although N. gonorrhoeae do not make CMP-Neu5Ac, the bacteria can utilize host CMP-Neu5Ac during infection to sialylate the outer membrane LOS, which increases the anionic charge of the LOS and confers greater resistance to complement-mediated killing by increased binding to human factor H (fH). N. gonorrhoeae was cultured on CMP-Neu5Ac containing GC-agar to determine the relative bactericidal activity of the linear compared to the TE-cyclized CPP on bacteria with sialylated LOS. The results (FIG. 10A-C) showed that the TE cyclized CPP at the 4 □M concentration was more potent than the linear CPP for all 3 strains and that it was also more bactericidal at the 20 □M concentration for the F89 and H041 strains (FIGS. 10A and C). Comparing bactericidal activity for sialylated (FIG. 10B-C) and non-sialylated (FIG. 8A-C) bacteria revealed differences at several concentrations. For example, at 4 μM tcCPP (TE cyclized CPP) was less active against sialylated (FIG. 10B) compared to non-sialylated F89 bacteria (FIG. 8B), but at 20 μM was more active against sialylated (FIG. 10C) compared to non-sialylated H041 bacteria (FIG. 8C). The results demonstrated that the CPPs have bactericidal activity for sialylated N. gonorrhoeae and that the tcCPP was more potent than the linear CPP (ICPP) regardless of sialylation state.

### 5. TE Cyclized CPP has More Potent Bactericidal Activity than Linear CPP in Presence of Human Endocervical, Cervical, and Monocytic Cells

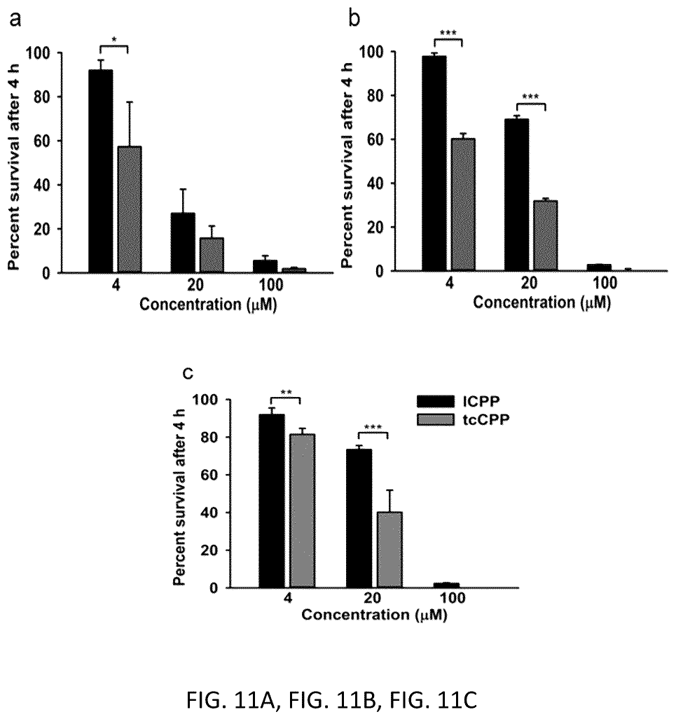

Bactericidal assays using the linear and TE cyclized CPPs were also performed with the FA1090 strain in the presence of three different types of human cells, End1/E6E7 endocervical epithelial cells, ME-180 cervical epithelial, and THP-1 monocytes. The results (FIG. 11) revealed that the TE cyclized CPP had more bactericidal potency for the FA1090 gonococci at the 20 M in the presence of the ME-180 and the THP-1 cell types and tended to have more potency for the End1/E6E7 cells. At the 4 □M concentration in the presence of the ME-180 and THP-1 cells the TE cyclized CPP was more potent for the FA1090 bacteria in the presence of all three cell types. Comparing the bactericidal activity for FA1090 without (FIG. 8A-C) and with (FIG. 11A-C) human cell types showed that the presence of the cells did reduce the potency of both linear and TE cyclized CPP as was expected. Nonetheless, these results demonstrate that the CPPs are bactericidal in the presence of human cells and, furthermore, provide additional evidence of the relative greater potency of the TE-cyclized compared to the linear CPP.

### 6. Lower TNF-α Levels Expressed by THP-1 Cells Infected with Gonococci Treated with the TE Cyclized Versus Linear CPP

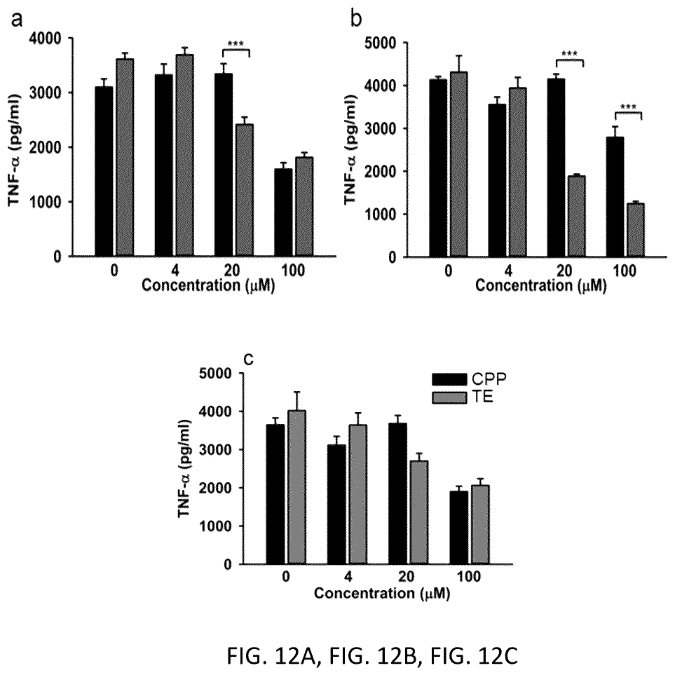

Treatment with the linear CPP reduced inflammatory signaling that was induced in THP-1 cells by killing the bacteria. To determine the relative effect of the TE cyclized CPP on inflammatory signaling, THP-1 cells were incubated with each of the 3 bacterial strains at a moi of 1 in the presence of the linear and TE cyclized CPP at 0, 4, 20, and 100 □M overnight. As shown in FIG. 12, TE-cyclized CPP treatment resulted in a statistically significant decrease (P<0.001) in TNF-□ expression relative to the linear CPP at the 20 □M concentration in THP-1 cells infected with the FA1090 (FIG. 12A), and at the 20 □M and 100 □M concentration in THP-1 cells infected with F89 (FIG. 12B). Relative to vehicle-only controls there were significant reductions in TNF-□ levels in bacteria treated with TE cyclized CPP in all bacteria at the highest concentration of 100 □M, and in the FA1090 and F89 bacteria treated with TE cyclized CPP at the 20 □M concentration. There was little effect of treatments at the lower concentration of 4 □M on TNF-□ levels likely due to the TNF-□ induction by LOS released in membrane blebs from dead or dying bacteria.

### 7. CPPs Exhibit Bactericidal Activity Against Three Neisseria Commensal Species

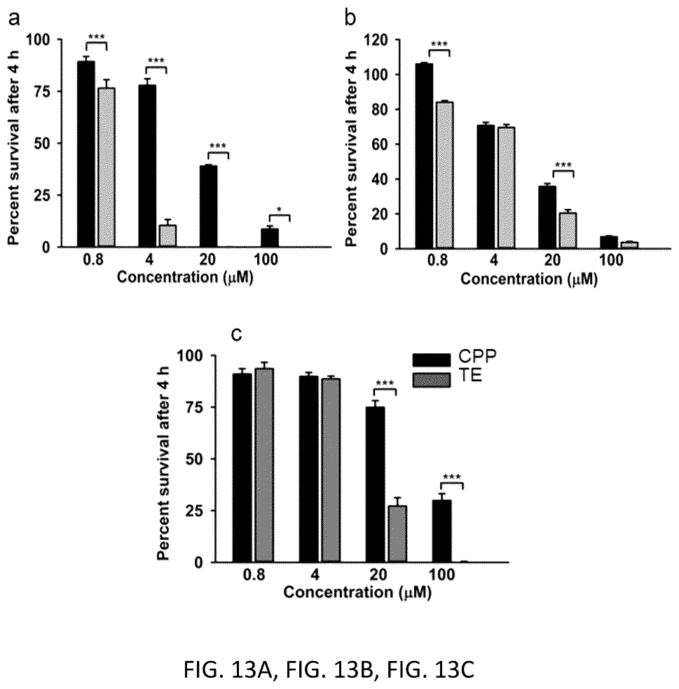

There are eight species of commensal Neisseria that can colonize the upper respiratory tract and are considered nonpathogenic although there are occasional reports of infection by the organism. Depending on the species the rates of carriage range from 3-40% of the population and a growing body of data show that commensal bacteria can serve as a reservoir of transferable antimicrobial resistance genes. Therefore, the bactericidal activity of the linear and TE cyclized CPPs were compared in strains of three commensal species, N. flavescens, N. lactamica, and N. subflava. The data showed that the TE cyclized CPP had significantly more potent bactericidal activity than the linear CPP for all three species at the 20 mM concentration and for N. flavescens and N. lactamica also at lower concentrations (FIG. 13). The sensitivity to the CPPs varied between the three species. N. flavescens (FIG. 13A) was the most sensitive and N. subflava (FIG. 13C) exhibited the least sensitivity to the TE cyclized CPP.

### 8. CPPs have Limited Activity Against Gram Positive Staphylococcus aureus

The sensitivity of a Gram positive species was also tested. Sensitivity of two strains of S. aureus to the linear and TE cyclized CPP was assessed to reveal potential for broad spectrum bactericidal activity. The results (Table 1) showed that the potency of the CPPs for S. aureus was markedly less than that of the N. gonorrhoeae or Neisseria commensal strains. There was no evidence of complete eradication of the S. aureus even at the 100 mM concentration.

TABLE 1
S aureus survival* after 4 h treatment.

| Strain | 8325-4 | Newman |
|---|---|---|
| ICPP (100 μM) | 130.5 (±2.4) | 67.4 (±9.2) |
| tcCPP (100 μM) | 88.0 (±5.5) | 37.1 (±4.8) |

*mean percentage of vehicle only control OD ± SEM

### 9. Discussion

Cyclization of a linear (R—X—R)₄ CPP using a TE bond increased the bactericidal potency for N. gonorrhoeae by a factor of approximately 10-fold with a similar increase in sensitivity observed with bacteria having sialylated LOS. The potency of a DS cyclized in the bactericidal assays was intermediate to that of the linear and TE cyclized CPP.

The classic use of CPPs, many of which are highly positively charged, is penetration of mammalian cell membranes for the delivery of cargo intracellularly and the original interest in the linear (RXR)₄ (SEQ ID NO:4) CPP was toward exploring its use as a vehicle for intracellular delivery of antibacterial antinucleotide morpholino oligomers. However, differentiation of AMPs and CPPs, which are both typified by interactions with cellular membranes, is not always clear and previous studies led to the observation of the antimicrobial activity of the linear CPP itself. Analyses of the antimicrobial activity of cationic peptides such as CPPs has shown the presence of lysine and especially arginine in highly-positively charged peptides enhances their seemingly contradictory capability of penetrating or disrupting nonpolar mammalian membranes. Exploiting the differences in relative affinity for mammalian versus bacterial membranes is thought to be a determinant of potency of AMPs. Compared to lysine the presence of arginine has been shown to confer more bactericidal activity and more selectivity for bacterial compared to mammalian cells because arginine can form two hydrogen bonds and has greater charge distribution.

The modes of interaction of AMPs with bacteria have been found to vary being characterized by a carpet model that is primarily detergent-like disruption of bacterial membranes, a barrel stave mode that is characterized by formation of amphipathic pores, and a toroidal model that is a combination of these two modes in which after aggregating on the surface, the peptides insert into the bacterial membrane and induce monolayer bending. However, other studies indicate that some AMPs may be passively uptaken and kill bacteria by binding to intracellular nucleic acids and proteins. Cyclizing AMPs, which can be formed by covalent bridges between side chains to produce what are called stapled peptides or by forming amide bonds between the amino and carboxyl termini, restrict the formation of secondary structures and is expected to enhance penetration of the bacterial cell wall and, thus, subsequent intracellular binding to intracellular components such as nucleic acids relative to binding to negatively charged outer membrane components such as the LOS. The data demonstrate the potential of cyclizing a guanidinium-rich peptide, which could be compared to a Velcro strip due to its ability to adhere to oppositely charged moieties on bacterial cell surfaces and to negatively charged nucleic acids and proteins to improve bactericidal activity of a peptide active against N. gonorrhoeae.

Sialylation of the FA1090 LOS did not prevent the CPPs from killing the bacteria although it did variously affect potency (FIG. 10). FA1090 1-81-S2 human challenge strain expresses lipid A that is phosphoethanolaminylated and that the oligosaccharide moiety of the LOS can be disialylated. The presence of Neu5Ac (sialic acid) on the bacterial LOS would increase its negative charge and, thus, increase the affinity of the positively charged CPPs for the LOS. There is evidence from studies of 6 a.a. cyclic positively charged AMPs that binding to the bacterial membrane may not be simply equated with bactericidal activity because if the initial binding of the AMPs to LPS in the membrane was too strong, potency was reduced due to less translocation of the AMPs into the lipid bilayer. The fact that LOS sialylation decreased the potency of the TE cyclized CP for FA1090 at the 20 μM concentration indicates that its mode of action relies at least partially on penetration of the bacterial membranes and/or inhibiting essential processes intracellularly rather than simple membrane disruption.

All three commensal Neisseria strains were sensitive to the TE cyclized CPP. N. subflava strain 52, which lacks pyrophosphoryl and phosphoethanolamine groups on the lipid A, was more susceptible than N. lactamica, which expresses both groups. Some commensal Neisseria have been reported to be highly resistant to antibiotic treatment and being capable of horizontal gene transfer to N. gonorrhoeae, can serve as a reservoir of MDR-conferring genes, The potential of the commensal bacteria to harbor resistance genes recently prompted a call to expand gonococcal surveillance to include resistance profiling and whole genome sequence of commensal Neisseria isolates. Perhaps although they are a component of normal microbiota in the oropharynx, bactericidal activity against the commensal Neisseria could be advantageous in this era of increasingly MDR gonorrhea.

There was virtually no cytotoxicity exhibited for any of the cell types by either the linear or the TE cyclized CPPs at 100 mM (FIG. 8A-C), which is approximately 10-fold higher than the gonococcal MBC50. In contrast, LL-37 exhibited significant cytotoxicity for the End1/E6E7 and ME-180 cells at much lower concentrations of 20 and 40 mM, albeit these concentrations were in the range of MBC for N. gonorrhoeae. Although the DS cyclized CPP was relatively the most cytotoxic of the three CPPs at the 100 and 200 mM concentrations tested, the percentage of dead cells compared to the Triton X-100 treated controls was less than 20%, which was similar to the cytotoxic levels observed in mouse macrophage RAW 264.7 cells from exposure to a 21 a.a. AMP with an intramolecular disulfide bond and a net charge of +6 that was isolated from Branchiostoma japonicum.

The structural significance of cyclization to the increased potency is supported by the results with the DS cyclized CPP that was introduced by adding two Cys residues. Compared to TE bonds, DS bonds can be more readily reduced entropically or through reduction by mammalian and bacterial NADPH and oxoreductases such as glutathione, which can occur both intra- and extracellularly. Interestingly, the DS compared to the TE cyclized CPP was more toxic to the mammalian cells. This implies some resistance of the bacteria to the toxicity of the DS cyclized CPP, which conceivably could be due to its induction of oxidative stress that perhaps the bacteria can more readily survive which is supported by the fact that it can also occur at the sites of infection due to host inflammatory responses.

In analyzing the activity of the CPPs we added serum to the culture media, tested bactericidal potency for N. gonorrhoeae grown in the presence of CMP-NANA to enable sialylation of the bacterial LOS and determined bactericidal activity in the presence of human cells. Testing in the presence of serum and host cells has been proposed as essential in evaluating therapeutic potential of AMPs because host-cell interactions such as binding to serum proteins such as serum albumin and other proteins could reduce their antimicrobial activity in vivo and, conversely, the presence of complement in serum could have antibacterial effects.

Currently, there are two cationic AMPs that are used clinically, polymyxin B and colistin (polymyxin E). The latter is administered as the inactive prodrug, colistin methanesulfonate that is converted to colistin in vivo. The systemic use of these long-standing antibiotics, which can give rise to dose-limiting toxicity, has reemerged as salvage treatment for MDR Gram-negative bacterial infections. However, in the last few years there also have been clinical trials of several peptides of synthetic or natural origin, including PL-5, POL7080, which is derived from protein 1, DPK-060, and the endogenous AMP LL-37, for treatment of bacterial infections.

The lack of highly effective resistance to the endogenous cationic AMPs, which are evolutionarily ancient, could be viewed as surprising when compared to the relatively rapid resistance that develops to most antibiotics, and it has been proposed that bacterial resistance mechanisms co-evolved with cationic AMPs as part of host-pathogen adaptation. Resistance mechanisms include inactivation by bacterial proteases, by binding of the endogenous cationic AMPs to secreted microbial proteins, and by active extrusion, such as by the MtrCDE MDR exporter in Neisseria gonorrhoeae and N. meningitidis. Another mechanism of resistance is the reduction of the anionic charge on the bacterial cell membrane, for example, by alteration of the negative-charge state of LPS or lipooligosaccharide by dephosphorylation or by derivatization with galactosamine and phosphoethanolamine (PEA). The widespread expression of the mer-1 plasmid, which encodes a PEA transferase that causes PEA modification of lipid A and confers polymyxin resistance as does the expression of PEA in lipid A of pathogenic Neisseria, has contributed to the global crisis of antibacterial resistance.

In summary, the DS and TE cyclized forms of the CPP had enhanced potency in bactericidal assays with all 3 strains of N. gonorrhoeae including the two MDR strains, F89 and H041, compared to the linear CPP. The TE cyclized CPP was more potent compared to the DS cyclized CPP in bactericidal assays with strain F89 and was more potent than the linear CPP in the presence of cervical and monocytic cells. This mirrored the greater reduction in expression of TNF-α by monocytes incubated with bacteria treated with the TE cyclized CPP compared to linear CPP. The TE cyclized CPP was bactericidal to gonococci with sialylated LOS.

### 10. Materials and Methods

i. Bacterial Strains

N. flavescens strain 4322, N. lactamica strain 1934, and N. subflava strain 52, were available from the collections of the late Dr. Herman Schneider of the Walter Reed Army Institute of Research and of the Neisseria Repository that was formerly housed at UC Berkeley. The 8325-4 and Newman are strains of S. aureus. The latter is β-hemolytic.

ii. Bacterial Survival Assays

Stock solutions of the CPPs were prepared in DMSO and aliquots were frozen at −80° C. Bacteria were grown overnight on Difco GC agar (Becton, Dickinson, Sparks, MD, USA) containing 1% BBL Isovitalex Enrichment (Becton, Dickinson), and then harvested and suspended in calcium- and magnesium-free PBS. Absorbance of the bacterial suspension at 595 nm was measured on a DU 730 spectrophotometer (Beckman Coulter, Pasadena, CA, USA) and used to serially dilute bacteria in RPMI 1640 media (Gibco, ThermoFisher Scientific, Pittsburgh, PA, USA) with 10% FBS. The survival assays were performed in 96-well plates (CellBIND, Corning, Glendale, AZ, USA). Each test well contained bacteria plus 200-μL total volume with 10-μL aliquots of diluted stock solutions of the CPPs or DMSO only in RPMI 1640 with 10% FBS. The 96-well plates were incubated at 37° C. in 5% CO₂, for 4 h, mixed gently, and then 10-μL aliquots were plated in duplicates on Difco GC agar plates. After overnight incubation of the agar plates, the colonies were counted and percent survival was reported for each test concentration relative to the DMSO-only control. To test the effect of sialylation on bactericidal activity of the CPPs, bacteria were grown overnight on Difco GC agar containing 1% BBL Isovitalex Enrichment and 10 μg/mL CMP-NANA (CMP-sialic Acid, Sigma-Aldrich, St. Louis, MO, USA).

iii. Cytotoxicity of CPPs for Human Cells

We analyzed the levels of lactate dehydrogenase (LDH) released from dying human End1/E6E7 epithelial endocervical cells1, ME-180 epithelial cervical2, and THP-1 monocytes due to cytotoxicity from exposure to the CPP structural analogs or LL-37 using the Cyquant LDH Cytotoxicity Assay Kit (Invitrogen ThermoFisher Scientific). The THP-1 cells were activated overnight by incubation with 10 ng/ml of phorbol myristate acetate (PMA; Sigma-Aldrich). After washing, cells were trypsinized, and then 1.0×10⁴ cells in 100-μL media were plated per well in 96-well plates. For the activated THP-1 and ME-180 cells, Opti-MEM (Gibco) with 1% FBS was used. For the End1/E6E7 cells Keratinocyte-Scrum Free Media (ThermoFisher Scientific) with supplements was used. After plating, the activated THP-1 cells were incubated for 4-5 h and the End1/E6E7 and ME-180-cells were incubated overnight. Next stock solutions of the CPPs or LL-37 were diluted in PBS and aliquots were added to wells to create final concentrations of 100 and 200 μM of the CPPs, or 20 and 40 μM of LL-37 in total volumes of 100 μL per well. Negative control wells were treated with vehicle only, which was 1% DMSO in media. Vehicle controls for LL-37 were wells with Opti-MEM with 1% FBS only. After adding the test reagents, plates were gently mixed and incubated at 37° C. and 5% CO₂ for 18 h. Positive control wells were treated with 10 μL of the Triton X-100 Lysis Buffer provided with the Cyquant kit according to the directions of the vendor. Then the levels of LDH in supernatants were quantified using a kit (Roche, Mannheim, DE, USA) with detection on an Epoch (Biotek Instruments, Winooski, Vermont, USA) microtiter plate reader at 490 nm with 680 nm subtracted. The results were reported as the percentage of LDH released from cells treated with LL-37 or CPP relative to LDH released from control cells treated with vehicle-only.

iv. Hemolysis Assay

The CPPs or the vehicle (DMSO and 0.1% Triton X-100), were diluted in PBS. Washed pooled sheep red blood cells (sRBCs; Rockland, R405-0050, 10% v/v) were diluted to 2.1% in PBS. Then 190 μL of the cell suspension was added to the wells of a 96-well plate (Corning CellBIND). Solutions (10 μL) containing the linear or DE or TE cyclized forms of the CPP at 4,000 or 2,000 μM or the DMSO and PBS vehicle were added to wells with sRBCs in triplicate. As a positive control three wells were treated with 10 μL of 2% Triton X-100. The 96 well plates were incubated at 37° C. and 5% CO₂ for 1 h, and then centrifugated at 1,000×g for 5 min. Supernatant (100 μL) was transferred to a new plate and then analyzed on the Epoch microtiter plate reader at 405-nm. Results are expressed as % hemolysis, which was calculated according to the following equation: % hemolysis=[(405 nm absorbance (Abs) of RBCs in the peptide solution-405 nm Abs in PBS)/(405 nm Abs in 0.1% Triton X-100-405 nm ABs in PBS]×100. Values of >25% hemolysis were defined as poor, 10-25% medium, and <10% as non-hemolytic according to recommended guidelines.

v. Bacterial Survival Assay in the Presence of Human Cells

Assays were performed in 96-well CellBIND plates with activated human THP-1 monocytic cells (ATCC), immortalized endocervical cells (End1/E6E7; ATCC) or immortalized cervical cells (ME-180; ATCC) (30,000-40,000 cells/well) in 180 μL of RPMI-1640 (10% FBS) for the THP-1 and the ME-180 cells, or Keratinocyte Serum Free Media (Keratinocyte SFM 1X; ThermoFisher Scientific) with supplements for the End1/E6E7 cells. After the cells adhered to the wells, the bacterial survival assay protocol described above was performed with the exception that after the 4 h incubation, 100-μL of 3% saponin was added to the 200 μL media in each well. Then the plate was incubated in 5% CO₂ at 37° C. for an additional 15 minutes, each well mixed gently by pipetting up and down, and then plated on GC agar plates for determination of CFUs as described above.

vi. Effect of CPP on Induction of TNF-α Expression from Gonococcal Infection of THP-1 cells

THP-1 human monocytic cells were cultured in RPMI 1640 with 10% FBS in 5% CO₂ at 37° C. The cells were activated by incubation in media with 10 ng/ml of PMA for 18 h. After washing with calcium- and magnesium-free PBS, the cells were trypsinized and plated (CellBIND, Corning) in RPMI-1640 with 10% FBS (1.0×10⁴ cells/well) per well. Gonococci grown overnight on Difco GC agar containing 1% BBL Isovitalex Enrichment, were suspended in PBS and the absorbance at 595-nm was determined. Gonococci were diluted in cell culture media and then added to the THP-1 cells at a moi of 1. Aliquots of CPP in RPMI 1640 with 10% FBS were created from DMSO stock solutions and added to the wells to achieve final concentrations of 4, 20, or 100 UM in total volumes of 200 L/well. Plates were incubated at 37° C. and 5% CO₂ overnight, and then supernatant from each test well was transferred to a fresh 96-well plate, scaled, and frozen at −80° until analysis. For detection of TNF-α in aliquots of the supernatants, a kit (Human TNF alpha Uncoated ELISA kit, Invitrogen, ThermoFisher Scientific) was used and the results were analyzed on an Epoch plate reader at 450-nm.

vii. Determination of Bactericidal Activity of CPPs for Staphylococcus aureus

S. aureus were grown overnight on Difco GC agar containing 1% BBL Isovitalex Enrichment. Using a sterile cotton swab, bacterial colonies were placed in suspension in GC broth containing 1% BBL Isovitalex. Absorbance of the bacterial suspension at 595 nm was determined to enable dilution to approximately 4×10⁶ cells/mL in GC broth. Aliquots of the suspension containing 8×10⁵ bacteria along with the CPPs, diluted from stock solutions in DMSO, were added in GC broth containing 1% BBL Isovitalex to make a total volume of 200 μL/well. Bacteria in GC broth with Isovitalex and 1% DMSO were added to vehicle-only control wells. Each test condition was plated in sextuplicates. Peripheral wells of the 96-well plate were filled with filtered deionized water to minimize edge effects. After incubation in 5% CO₂ at 37° C. for 18 h, plates were analyzed at 600-nm on an Epoch microtiter plate reader. The percentage inhibition of each sample was determined relative to the absorbance of the blank (GC broth only) and the vehicle-only controls. Any inhibition found to be less than 30% relative to the vehicle-only controls was further verified by plating the suspension on GC agar containing 1% Isovitalex, followed by overnight incubation and then counting to assess bacterial viability. If no growth was observed on the plate, then that test concentration was determined to be inhibitory.

viii. Statistical Analyses

SigmaPlot version 12.5 (Systat Software, San Jose, CA) was used to perform statistical analyses. Multigroup comparisons were performed using one-way analysis of variance with post hoc tests. Significance was defined as P<0.05 for all comparisons.

Those skilled in the art will recognize, or be able to ascertain using no more than routine experimentation, many equivalents to the specific embodiments of the method and compositions described herein. Such equivalents are intended to be encompassed by the following claims.

We claim:

1. A cyclic cell penetrating peptide (cCPP) comprising a 6-aminohexanoic-spaced oligoarginine ((R-Ahx-R)₄).

2. The cCPP of claim 1, further comprising cysteines on the N- and C-terminal ends of (R-Ahx-R)₄.

3. The cCPP of claim 1-2, wherein the peptide comprises a disulfide bridge.

4. The cCPP of claim 1-2, wherein the peptide comprises a thioether bridge.

5. A cCPP comprising the amino acid sequence of CRXRRXRRXRRXRC (SEQ ID NO: 1), wherein X is aminohexanoic acid.

6. The cCPP of claim 5, wherein the peptide comprises a disulfide bridge.

7. A cCPP comprising the amino acid sequence of RXRRXRRXRRXRC (SEQ ID NO: 2), wherein X is aminohexanoic acid.

8. The cCPP of claim 7, wherein the peptide comprises a thioether bridge.

9. A method of treating a subject having a N. gonorrhoeae infection comprising administering one or more of the cCPPs of any one of claims 1-8 to the subject.

10. A method of killing N. gonorrhoeae comprising administering one or more of the cCPPs of any one of claims 1-8 to the N. gonorrhoeae.

11. The method of claim 10, wherein the N. gonorrhoeae is in vitro or in vivo.

12. The methods of 9-11, further comprising administering a therapeutic known to counter Gram-negative bacterial infections.

13. A method of reducing inflammatory signaling from cells infected with N. gonorrhoeae comprising administering one or more of the cCPPs of any one of claims 1-8 to the cells infected with N. gonorrhoeae.
